(19) United States
(12) Patent Application Publication
(10) Pub. No.: US 2024/0299438 A1
(43) Pub. Date: Sep. 12, 2024

(54) METHOD FOR DIAGNOSIS AND TREATMENT OF AUTISM SPECTRUM DISORDER ON BASIS OF ACTIVITY REGULATION MECHANISM OF DORMANT NEURAL STEM CELL

(71) Applicant: DONGGUK UNIVERSITY INDUSTRY-ACADEMIC COOPERATION FOUNDATION, Seoul (KR)

(72) Inventors: Jongpil KIM, Seoul (KR); Hongwon KIM, Gyeonggi-do (KR); Byounggook CHO, Gyeonggi-do (KR)

(73) Assignee: DONGGUK UNIVERSITY INDUSTRY-ACADEMIC COOPERATION FOUNDATION, Seoul (KR)

(21) Appl. No.: 18/270,915

(22) PCT Filed: Dec. 31, 2021
(86) PCT No.: PCT/KR2021/020344

§ 371 Date: Jul. 5, 2023

(30) Foreign Application Priority Data
Jan. 5, 2021 (KR) 10-2021-0000957

(51) Int. Cl. A61K 31/7105; A01K 67/027; A61K 31/5355; A61P 25/18; G01N 33/50

(52) U.S. Cl. CPC A61K 31/7105; A01K 67/027; A61K 31/5355; A61P 25/18; G01N 33/5058; G01N 33/5073; A01K 2207/05; A01K 2207/20

(57) ABSTRACT

Provided are diagnosis and treatment methods of autism spectrum disorder, based on an activity regulation mechanism of quiescent neural stem cells, and use thereof.

### BACKGROUND OF THE INVENTION

### 1. Field of the Invention

The present disclosure relates to diagnosis and treatment methods of autism spectrum disorder, based on an activity regulation mechanism of quiescent neural stem cells, and use thereof.

### 2. Description of the Related Art

Among neuropsychiatric disorders, autism is known as a neurodevelopmental disorder caused by abnormal development of brain nerves from infancy, and the disease is known to be induced due to mutations in various genes or environmental factors.

Shank3 gene is a representative genetic cause of autism. In addition, Chd8, Pten, Clock19, etc. are known to have roles in development of the disease; particularly, they are known to cause problems in synaptic function and development of nerve cells. For example, it has been reported that pathological symptoms appear due to abnormal expression of HOMER and SAPAP3, which are synaptic component proteins, and AMPA and NMDA receptors due to morphological and functional deterioration of excitatory synapses. Until now, studies have been confined to identifying symptoms of synaptic imbalance observed in excitatory and inhibitory neurons rather than understanding the fundamental pathogenesis in the process of neurodevelopment. Accordingly, there is an urgent need to develop a new treatment technology based on the fundamental pathogenesis for the treatment of autism. In addition, even though various studies have been conducted on the cause of autism disorder, there are many shortcomings in explaining the behavioral analysis aspects related to the synaptic function of neurons.

Therapies currently used for the treatment of autism include pharmacotherapy and cognitive behavioral therapy. Pharmacotherapy plays a role in controlling the abnormal action of neurotransmitters and preventing and delaying neurotoxicity and cell death by blocking neurotransmitters secreted from nerve cells (dopamine, serotonin, vasopressin, norepinephrine, etc.) or receptors thereof. Examples thereof include antidepressants/SSRIs, antipsychotics, anticonvulsants, stimulants, etc. However, all of these drugs are used for the purpose of alleviating the symptoms observed in patients, and their fundamental therapeutic effects on autism are limited.

In addition, cognitive behavioral therapy aims to promote normal development such as intelligence, language development, sociability, communication, cognition, etc., and is able to alleviate symptoms through systematic treatment in the early stages of onset and maximize the therapeutic effect in combination with pharmacotherapy. However, neurodevelopmental disorders such as autism, etc. have a prevalence of 2 to 5 per 10,000 children under the age of 12, and it is difficult to diagnose the disease in early childhood.

The present inventors have endeavored to identify target cell groups and specific disease factors that play a key role in the onset of autism, and as a result, they found that the dormancy of quiescent neural stem cells (quiescent NSCs, qNSCs) is closely related to autism, and autistic behaviors are alleviated as a result of treatment with a substance inhibiting the dormancy thereof, thereby completing the present invention.

### SUMMARY OF THE INVENTION

An object of the present disclosure is to provide a pharmaceutical composition for preventing or treating autism spectrum disorder, the pharmaceutical composition including a dormancy inhibitor of quiescent neural stem cells (qNSCs).

Another object of the present disclosure is to provide a method of preventing or treating autism spectrum disorder, the method including inhibiting dormancy of quiescent neural stem cells (qNSCs) in an individual excluding humans.

Still another object of the present disclosure is to provide a method of screening for a therapeutic agent for autism spectrum disorder, the method including the steps of (a) treating quiescent neural stem cells with a candidate therapeutic agent for autism spectrum disorder; and (b) measuring dormancy of the neural stem cells.

Still another object of the present disclosure is to provide a composition for diagnosing autism spectrum disorder, the composition including an agent capable of measuring dormancy of quiescent neural stem cells.

Still another object of the present disclosure is to provide a method of providing information for diagnosis of autism spectrum disorder, the method including the steps of (a) measuring dormancy of quiescent neural stem cells in an individual suspected of developing autism spectrum disorder; and (b) comparing the measured level with that in a normal individual.

Still another object of the present disclosure is to provide a method of preparing an autism spectrum disorder animal model, the method including the step of increasing dormancy of quiescent neural stem cells in an animal excluding humans.

Still another object of the present disclosure is to provide use of the dormancy inhibitor of quiescent neural stem cells in the prevention or treatment of autism spectrum disorder.

Still another object of the present disclosure is to provide use of the agent capable of measuring dormancy of quiescent neural stem cells in the diagnosis of autism spectrum disorder.

### BRIEF DESCRIPTION OF THE DRAWINGS

FIG. 1 (A) Data showing nine distinct clusters with cell-type identities as determined by expression of specific markers. (B) Graph showing percentages of respective cell groups. (C) Neural stem cells in the SVZ region identifying 4 sub-clusters across conditions. (D) Cell groups expressing neural stem cell-type-specific markers identifying each cluster. (E) Comparison to previous datasets; Left, 307 genes contained within the qNSC cluster (highest in qNSCs); right, 130 genes from the aNSC cluster (highest in aNSCs). The clusters were compared to a previously reported qNSC microarray. P values according to the hypergeometric test. (F) Quantification of neural stem cells of the indicated types. (G) Pseudo-time trajectory of cell density. (H) Cell cycle scoring analysis in neural stem cells of control and Shank3 KO mice. (I) Ratio of assigned cell cycles of neural stem cells.

FIG. 2 validates the existence of adult neurogenesis by immunofluorescence for Sox2, Cd133 (A), DIx2 (B), and DCX (C) in the subventricular zone of control and Shank3 mutant mice. Scale bar=20 μm. (D) Percentages of Cd133+, Dlx2+, and DCX+ Sox2 positive cells in control and Shank3 KO mice. (E and F) Results of immunostaining of quiescent neural stem cells in the SVZ of control and Shank3 KO mice. Yellow arrows indicate qNSCs and white arrow indicate aNSCs. Scale bar=10 μm. (G) Percentages of GFAP+Sox2+Dlx2− qNSCs and GFAP+Sox2+Id2+ qNSCs among the total Sox2+ cells. (H) Percentages of GFAP+Sox2+EGFR− qNSCs and GFAP+Sox2+EGFR+ aNSCs among the total Sox2+ cells. (I) Percentages of GFAP+Blbp+BrdU+ cells in DG of control and Shank3 KO mice. (J and K) Results of immunofluorescence for DCX+ and Psa-ncam+ cells (J) and GFAP+Blbp+BrdU+ cells (K) in the SGZ of control and Shank3 KO mice. Scale bar=20 μm. (L and M) Immunostaining of quiescent neural stem cells in DG of control and Shank3 KO mice. Yellow arrows indicate qNSCs. Scale bar=20 μm. (N) Percentages of GFAP+Sox2+NeuN− and GFAP+Blbp+NeuN− qNSCs in the SGZ of control and Shank3 KO mice. (O) Results of immunostaining of the qNSCs and aNSCs expressing GFAP::GFP in the SVZ and DG of Shank3 KO mice into which GFP expressed by GFAP promoter was introduced. Scale bar=20 μm. (P) Percentages of GFAP::GFP+Sox2+Dlx2− qNSCs and GFAP::GFP+Blbp+NeuN− qNSCs. (Q) Immunostaining and percentages of GFAP::GFP+Sox2+Ki67− qNSCs and GFAP::GFP+Sox2+Ki67+ aNSCs in the SVZ of Shank3 KO mice. (R) Immunostaining and percentages of GFAP::GFP+Sox2+Ki67− qNSCs and GFAP::GFP+Sox2+Ki67+aNSCs in the DG of Shank3 KO mice.

FIG. 3 (A) Monocle pseudotime analysis of qNSC-to-primed qNSCs (primed qNSCs) according to conditions of control and Shank3 KO mice. (B) Results of heatmap of differentially expressed genes most significantly associated with pseudotime in quiescent neural stem cell groups. Cell fate 1 indicates a normal qNSCs activation state, and Cell fate 2 indicates an abnormal Shank3 KO qNSC state. (C) Data showing expression of Kmt2a, Hes1, and Ascl1 genes in qNSCs and primed qNSCs. (D and E) GSEA data showing significance of Kmt2/MII target gene (D) and H3K4me3 (E) target gene expression in Shank3 KO cells. (F) Quantification data of the number of qNSCs with increased H3K4me3 in the SVZ (top) and DG (bottom) of Shank3 KO mice. (G) Immunofluorescence staining showing nuclear translocation of β-catenin in GFAP+Sox2+ qNSCs from control and Shank3 KO mice. (H) ChIP-PCR quantitative analysis showing the binding of β-catenin protein at the Kmt2a promoter region (P2) containing the TCF/LEF binding motif. (I) ChIP-PCR quantitative analysis of the binding of Kmt2a protein at the Hes1 promoter region (P1) containing the TCF/LEF binding motif. (J) Heatmap of chromatin accessibility at TSS (transcription start site) as a result of ATAC-sequencing analysis of quiescent stem cells. (K) Chromatin accessibility of Kmt2a (top), Hes1 (bottom) promoter region in control and Shank3 KO conditions. (L) Chromatin accessibility of Ascl1 promoter region in control and Shank3 KO conditions.

FIG. 4 (A) Results of immunofluorescence for Sox2+Dlx2+ aNSCs in the SVZ of control and Shank3 KO mice treated with Kmt2a−shRNA. (B) Quantification data of Sox2+Dlx2+ aNSCs treated with Kmt2a−shRNA. (C) Quantification of BrdU+ NSCs among SVZs treated with Kmt2a−shRNA after a 3 hr BrdU treatment. Control and Shank3 KO mice treated with Kmt2a−shRNA were injected intraperitoneally with a daily dose of BrdU (100 mg/kg/day). (D) Immunofluorescence analyses of GFAP+Sox2+Dlx2− qNSCs in the SVZ of control and Shank3 KO mice treated with Kmt2a-shRNA. Scale bar=10 μm. (E) Quantification results of GFAP+Sox2+Dlx2− qNSCs after treatment with Kmt2a−shRNA. (F) Immunofluorescence for GFAP+Sox2+NeuN− qNSCs in the SGZ of control and Shank3 KO mice treated with Kmt2a−shRNA. Scale bar=20 μm. (G) Quantification results of GFAP+Sox2+NeuN− qNSCs after treatment with Kmt2a−shRNA. (H) Immunofluorescence analyses of Gfap+Nestin+H3K4me3+ qNSCs in the SGZ of control and Shank3 KO mice treated with Kmt2a− and Setd2− shRNA. Scale bar=10 μm. (I) Quantification results of Gfap+Nestin+H3K4me3+ qNSCs after treatment with Kmt2a− and Setd2−shRNA. (J) Results of representative heat maps showing the time spent in 3 chambers as a mouse behavior test to assess the social behavior in Control and Shank3 KO mice treated with Kmt2a− and Setd2− shRNA. (K and L) ratios of the time spent exploring (K) either nonsocial stimuli (NS) or social stimuli (Soc) and social preference index (L) of control and Shank3 KO mice treated with Kmt2a−shRNA.

FIG. 5 (A) Immunostaining results of quiescent neural stem cells in the SVZ of control and Shank3 KO mice injected with saline, OICR-9429 (3 mg/kg, once/day, 3 days), romidepsin (0.25 mg/kg, once/day, 3 days), and clozapine (5 mg/kg, once/day, 3 days). The yellow arrows indicate GFAP+Sox2+Dlx2− qNSCs. Scale bar=20 μm. (B) Immunostaining results of quiescent neural stem cells in the DG of control and Shank3 KO mice injected with saline, OICR-9429 (3 mg/kg, once/day, 3 days), romidepsin (0.25 mg/kg, once/day, 3 days), and clozapine (5 mg/kg, once/day, 3 days). The yellow arrows indicate GFAP+Sox2+NeuN− qNSCs. Scale bar=20 μm. (C-E) Quantification results of FAP+Sox2+Id2+ (C) and GFAP+Sox2+Dlx2− (D) qNSCs in the SVZ and GFAP+Sox2+NeuN− (E) qNSCs in the DG. (F) Quantification results of BrdU+ NSCs in the SVZ injected with saline, OICR-9429, romidepsin, and clozapine. Control and Shank3 KO mice injected with each drug were injected intraperitoneally with a daily dose of BrdU (100 mg/kg/day). (G) Quantification results of DCX+ NPCs in the DG of control and Shank3 KO mice injected with each drug. (H) Immunofluorescence analysis of GFAP+,Sox2+,BrdU+ aNSCs in the SVZ of control and Shank3 KO mice injected with each drug. Scale bar=100 μm. (I) Quantification results of BrdU+ cells in the SVZ of saline- or OICR-9429-treated control and Shank3 mice at different time points after TMZ injection. (J) Analysis of protein expression levels of synaptic proteins and glutamate receptor subunits from control or Shank3 KO mice injected with saline or OICR-9429. (K) Results of representative heat maps showing the time spent in 3 chambers to assess the social interactions of control and Shank3 KO mice treated with saline, OICR-9429, romidepsin, clozapine, and VPA. (L-O) Social preference index percentages (Soc vs. NS ratio) in Shank3 KO mice injected with OICR-9429 (L), romidepsin (M), clozapine (N), and VPA (O) for 28 days.

FIG. 6 (A) Immunostaining results of GFAP+,SOX2+,ID2+ and GFAP+,SOX2+,NESTIN+ quiescent neural stem cells (radical glia cell, RGCs) from control or SHANK3-deficient iPSCs. White arrows indicate human RGCs. Scale bar=20 μm. (B) Quantification results of GFAP+,SOX2+,I D2+ RGCs with radial morphology. (C) Quantification results of GFAP+,SOX2+,NESTIN+ RGCs with radial morphology. (D) Immunostaining of GFAP+,SOX2+,EGFR+ aNSC and K167+ proliferating cells. Scale bar=20 μm. (E) Quantification results of GFAP+,SOX2+,EGFR+ aNSCs from control or SHANK3-deficient RGCs. (F) Quantification results of K167+ proliferating cells from control or SHANK3-deficient RGCs. (G) Immunofluorescence analysis of GFAP+,SOX2+,NESTIN+ RGCs from control or SHANK3-deficient NSCs treated with KMT2A-shRNA. The white arrow indicates cells with radial morphology. Scale bar=20 μm. (H) Quantification results of the number of GFAP+,SOX2+,NESTIN+ RGCs with radial morphology and GFAP+,SOX2+,NESTIN+ RGCs with nonradial morphology treated with KMT2A-shRNA. (I and J) Western blot analysis of synaptic proteins and glutamate receptor subunits in control or SHANK3-deficient neuron-derived iPSCs treated with OICR-9429. (K and L) Western blot analysis of synaptic proteins and glutamate receptor subunits treated with romidepsin and clozapine. (M) UMAP plot of single cell RNA-seq data of human radial glia cells of Prefrontal cortex (PFC) and anterior cingulate cortex (ACC) from research results of Velmeshev et al. (N) Bar graph showing ratio of radial glia cells in PFC and ACC of control and autism patient. (O) Radial glia cells in PFC and ACC identifying 2 sub-clusters under control and autism patient conditions. (P) Results of comparison and analysis of similarity in the gene expression patterns between mouse and human. The closer to red, the higher the significance. (Q) Heatmap showing genes upregulated in quiescent neural stem cells of autism patients, as compared to quiescent neural stem cells of control. (R) Plots showing the expression of Kmt2a, Hes1, and Ascl1 genes, beta catenin, and quiescent neural stem cell markers in quiescent neural stem cells of control and autism patient.

FIG. 7 (A) Immunofluorescence analysis of Gfap+,Nestin+,H3K4me3+ qNSCs in the SGZ of control and Shank3 KO mice treated with shRNA against Kmt2c which is a histone modifier of H3K4me3. Scale bar=10 μm. (B) Quantification of Gfap+,Nestin+,H3K4me3+ qNSCs treated with Kmt2c-shRNA. (C) Results of assessing the social behaviors of Control and Shank3 KO mice treated with Kmt2c-shRNA. (D) Representative heat maps showing the time spent in 3 chambers to assess the social behaviors in Control and Shank3 KO mice treated with Kmt2c-shRNA. (E) Graphs showing social preference index ratios of nonsocial stimuli (NS) or social stimuli (Soc) of control and Shank3 KO mice treated with Kmt2c-shRNA.

### DETAILED DESCRIPTION OF THE PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

Further, those skilled in the art will recognize, or be able to ascertain using no more than routine experimentation, many equivalents to the specific embodiments of the disclosure described herein. Further, these equivalents should be interpreted to fall within the present disclosure.

An aspect of the present disclosure provides a pharmaceutical composition for preventing or treating autism spectrum disorder, the pharmaceutical composition including a dormancy inhibitor of quiescent neural stem cells (qNSCs).

As used herein, the term “quiescent neural stem cells (qNSCs)” refers to neural stem cells in a dormant state. Specifically, the dormant state may be at G0 or G1 phase of the cell cycle.

It is known that most adult neural stem cells exist in the dormant state, and they are distributed in the subventricular zone (SVZ) around the ventricle and in the subgranular zone (SGZ) of the hippocampus in the brain. On the other hand, adult neural stem cells are in the dormant state, and differentiate into neurons or play a role in producing neural stem cells through self-renewal, and therefore, quiescent neural stem cells are also called super neural stem cells. The neurodevelopmental process starting from the quiescent neural stem cells plays an important role in providing the brain with new brain cells.

As used herein, the “increase of dormancy” includes all of an increase in the number of neural stem cells in the dormant state, inhibition or reduction of quiescent neural stem cell activation, a decrease in the number of active stem cells, and an increase in the transition of active stem cells into the dormant state. Similarly, the “inhibition of dormancy” includes all of a decrease in the number of neural stem cells in the dormant state, an increase of quiescent neural stem cell activation, a decrease in the number of active stem cells, and an inhibition or reduction in the transition of active stem cells into the dormant state.

In one embodiment of the present disclosure, it was confirmed that the increase in the dormancy of quiescent neural stem cells (quiescent NSCs, qNSCs), compared to a normal level, is closely related to autism.

In one embodiment, in the present disclosure, inhibition of dormancy of quiescent neural stem cells may be inhibition of modification of histone proteins of neural stem cells. In other words, the dormancy inhibitor of quiescent neural stem cells may be a histone protein modification inhibitor.

Histone proteins are present in the core of nucleosomes, and consists of four core histones (H3, H4, H2A, H2B). In the present disclosure, the histone protein may be an H3 protein.

Modifications of histone proteins include methylation, phosphorylation, acetylation, ubiquitylation and sumoylation. In the present disclosure, the modifications of histone proteins may be methylation of histones. Methylation of histones may occur by histone methyltransferase (HMT), from which lysine residues or arginine residues of histones may be methylated.

On the other hand, when a lysine residue is methylated, each hydrogen of NH³⁺ is replaced with a methyl group, resulting in methylation. Thus, methylation includes mono-, di-, or tri-methylation.

In the present disclosure, the methylation of histones may be methylation of lysine residues. Specifically, the lysine residues may include lysine residues 4 (H3K4), 9 (H3K9), 27 (H3K27), and 79 (H3K79) on the H3 histone protein, specifically H3K4. More specifically, in the present disclosure, the methylation may be trimethylation of H3K4.

In the present disclosure, the inhibition of methylation includes a decrease, reduction, or inhibition of the activity of methyltransferase, or substitution of a residue, to which a methyl group is transferred by methyltransferase, with an amino acid other than lysine or arginine, or an increase in the activity of demethylase. Specifically, methylation may be inhibited by reducing the methyltransferase activity.

In the present disclosure, the reduction in the protein activity is a concept that includes both cases where the activity is weakened or absent, and may be used interchangeably with terms such as inactivation, deficiency, down-regulation, reduce, and attenuation, etc. This may also include a case where the activity is decreased or eliminated by variation of the gene encoding the protein, etc., a case where the protein activity level and/or concentration (expression level) is low due to inhibition of the expression of the gene or inhibition of translation, etc., a case where the gene is not expressed at all, and/or a case where the protein activity is not exhibited even when expressed.

In one embodiment, the inhibition of dormancy of quiescent neural stem cells of the present disclosure may be inhibition of activity of histone lysine methyltransferase of neural stem cells. In other words, the dormancy inhibitor of quiescent neural stem cells may be a histone lysine methyltransferase inhibitor.

As used herein, the “histone lysine methyltransferase (histone methyltransferase)” is an enzyme that performs methylation of lysine (K) residues on histone and is also called KMT. In the present disclosure, the lysine methyltransferase may be selected from Kmt2a, Kmt2b, Kmt2c, Kmt2f, Kmt2g, ASH, and Sc/Sp, specifically, one or more proteins selected from Kmt2a and Kmt2c.

As used herein, the “Kmt2a”, also called “histone-lysine N-methyltransferase 2A”, “ALL-1”, or “MLL1”, refers to a protein encoded by KMT2A gene. As used herein, the “Kmt2c”, also called “lysine methyltransferase 2C”, or “MLL3”, refers to a protein encoded by KMT2C gene. The Kmt2a and Kmt2c have lysine 4 (H3K4) methylation activity on H3 protein.

In one embodiment, the dormancy inhibitor of quiescent neural stem cells of the present disclosure may be one or more materials selected from OICR-9429, Kmt2a inhibitor, and Kmt2c inhibitor.

As used herein, the “Kmt2a inhibitor” or “Kmt2c inhibitor” is used to collectively refer to all of agents capable of reducing activity of the protein or expression of a gene encoding the same. This may be used without limitation in the form thereof, such as a compound, a natural extract, a chemical substance, a nucleic acid, a peptide, a virus, or a vector containing the nucleic acid, etc.

Specifically, the Kmt2a or Kmt2c inhibitor may be, but is not limited to, an interfering RNA, a ribozyme, a DNAzyme, a PNA (peptide nucleic acids), an antisense oligonucleotide, a peptide, an antibody, or an aptamer which is specific to the gene encoding the protein.

As used herein, the term “interfering RNA (short interfering RNA)” refers to a double-stranded RNA capable of inducing RNAi that inhibits gene activity. Specifically, the interfering RNA may be miRNA, siRNA, shRNA, etc. capable of suppressing the expression of KMT2A or KMT2C, but is not limited thereto. For example, siRNA obtained by chemical synthesis, biochemical synthesis, or in vivo synthesis, or double-stranded RNA of 10 nt or longer obtained by degrading double-stranded RNA of about 40 bases or more in vivo may be used.

As used herein, the term “microRNA (miRNA)” refers to a small RNA that plays a role in regulating gene expression in organisms, and is a small RNA containing 20 to 25 nucleotides that is able to play an important regulatory role in the gene expression process through the inhibition of target mRNA translation by complementary base pairing with the target mRNA 3′UTR (untranslated region). The microRNA plays an important role in cell functions including proliferation, differentiation, and apoptosis, etc., and is an evolutionarily conserved regulator present in all animals. Some microRNAs are known to cause regulation of gene expression through epigenetic regulatory mechanisms related to their promoter regions (histone modification, DNA methylation, etc.).

As used herein, the “siRNA” and “shRNA” is a nucleic acid molecule that is able to mediate RNA interference or gene silencing, and may be used as an efficient gene knockdown method or gene therapy method because it is able to suppress expression of a target gene. shRNA is a hairpin structure formed by binding between complementary sequences within a single-stranded oligonucleotide, and in vivo, the shRNA is cleaved by a dicer to form a double-stranded oligonucleotide siRNA which is a small RNA fragment of 21 nucleotides to 25 nucleotides in size, and may specifically bind to mRNA with a complementary sequence to suppress expression thereof. Therefore, which means of shRNA and siRNA to use may be determined by those skilled in the art, and when the mRNA sequences targeted thereby are identical, similar expression reduction effects may be expected.

As used herein, the term “antisense oligonucleotide” refers to DNA, RNA, or derivatives thereof containing a nucleic acid sequence complementary to a specific mRNA sequence, which inhibits translation of mRNA into a protein by binding to a complementary sequence in mRNA. The antisense oligonucleotide sequence of the present disclosure may refer to a DNA or RNA sequence that is complementary to mRNA of KMT2A or KMT2C and capable of binding to the mRNA. This may inhibit translation of the mRNA, translocation into the cytoplasm, maturation or other activities essential for all other overall biological functions.

As used herein, the “ribozyme” is a catalytic RNA molecule capable of degrading a nucleic acid molecule with a sequence which is completely or partially homologous to the sequence of the ribozyme. A ribozyme transgene may be designed, which encodes an RNA ribozyme that specifically pairs with a target RNA and cleaves the phosphodiester backbone at a specific location, thereby functionally inactivating the target RNA. In carrying out this cleavage, the ribozyme is not itself altered, and is thus capable of recycling and cleaving other molecules. The ribozyme may be directly targeted to cells in the form of an RNA oligonucleotide incorporating the ribozyme sequence, or may be introduced into the cell as an expression vector encoding a desired ribozyme RNA. The ribozyme may be generally used and applied in the same manner as described for antisense oligonucleotides.

As used herein, the “DNAzyme” is a catalytic DNA molecule that cleaves a single-stranded RNA, which is highly selective for target RNA sequences and thus may be used to down-regulate specific genes by targeting messenger RNA.

As used herein, the “aptamer” is a single-stranded oligonucleotide with a size of about 20 nucleotides to about 60 nucleotides, and refers to a kind of polynucleotide composed of single stranded nucleic acids (DNA, RNA, or modified nucleic acid) which has a stable tertiary structure as it is and is able to bind to a target molecule with high affinity and specificity. The aptamer may inhibit activity of a given target molecule by binding to the given target molecule. The aptamer may be RNA, DNA, modified nucleic acid or a mixture thereof, and may also be linear or cyclic.

In one embodiment of the present disclosure, it was confirmed that autism-related behaviors were improved by administering shRNA against Kmt2a and Kmt2c coding genes involved in H3K4 methylation to the subventricular zone (SVZ) region, which is abundant in neural stem cells, and the subgranular zone (SGZ) of the hippocampus in autism animal models. In another embodiment of the present disclosure, it was also confirmed that H3K4 trimethylation was inhibited by administering OICR-9429, which is a Kmt2a inhibitor, and accordingly, autism-related behaviors were improved.

In one embodiment, the inhibition of dormancy of quiescent neural stem cells in the present disclosure may include any one or more selected from inhibition of Hes1 expression; and an increase in an Ascl1 expression level in quiescent neural stem cells, but is not limited thereto.

As used herein, the “autism spectrum disorder” is, also called “autism”, a pervasive developmental disorder (PDD) and a neurodevelopmental disorder characterized by deficits in social interaction and communication, and repetitive and stereotyped behavior. The autism spectrum disorder appears in different forms and varies in severity depending on the individual, and includes one or more selected from the group consisting of autistic disorder, pervasive developmental disorder (PDD), deficits in social interaction and communication, repetitive and stereotyped behaviors, Rett's disorder, childhood disintegrative disorder, Asperger's syndrome, obsession disorder, obsessive compulsive disorder, and pervasive developmental disorder not otherwise specified (PDD-NOS).

The pharmaceutical composition of the present disclosure may include a pharmaceutically acceptable carrier.

The “pharmaceutically acceptable carrier” may refer to a carrier or diluent that does not abrogate biological activity and properties of a compound to be injected while not irritating living organisms. The type of carrier applicable in the present disclosure is not particularly limited, and any carrier may be used as long as it is commonly used in the art and pharmaceutically acceptable. Non-limiting examples of the carrier may include saline, sterile water, Ringer's solution, buffered saline, an albumin injection solution, a dextrose solution, a maltodextrin solution, glycerol, ethanol, etc. These may be used alone or in combination of two or more thereof.

Another aspect of the present disclosure provides a method of preventing or treating autism spectrum disorder, the method including inhibiting dormancy of quiescent neural stem cells (qNSCs).

As used herein, the term “preventing” means all of the actions by which the occurrence of autism spectrum disorder is restrained or retarded.

As used herein, the term “treating” means all of the actions by which the symptoms of autism spectrum disorder have taken a turn for the better or been modified favorably.

Specifically, the “preventing or treating autism spectrum disorder” may be achieved by inhibiting dormancy of quiescent neural stem cells. As described above, the inhibition of dormancy may be achieved by reducing the H3K4 trimethylation level in neural stem cells, by reducing the activity of histone lysine methyltransferase, and/or by reducing the expression level of genes encoding the same.

In the prevention or treatment method of the present disclosure, the dormancy inhibitor of quiescent neural stem cells may be administered parenterally.

In one embodiment of the prevention or treatment method of the present disclosure, the dormancy inhibitor may be administered to the subventricular zone (SVZ) region or the hippocampus subgranular zone (SGZ) in an individual, but is not limited thereto.

The “individual” of the present disclosure may refer to all animals including humans. The animals may be mammals such as mice, cows, horses, sheep, pigs, goats, camels, antelopes, dogs, and cats as well as humans. In addition, the animals may refer to animals other than humans, but are not limited thereto, and includes animals that are likely to develop or have developed autism spectrum disorder.

The “administering” of the present disclosure means introducing the composition of the present disclosure to an individual by any suitable method. The administration route of the present disclosure may be administered through any general route as long as it may reach a target tissue. In the treatment method of the present disclosure, the route of administration of the dormancy inhibitor of neural stem cells is not particularly limited, but the dormancy inhibitor may be administered through a route such as intraperitoneal administration, intravenous administration, intramuscular administration, subcutaneous administration, intradermal administration, oral administration, intranasal administration, intrapulmonary administration, intrarectal administration, etc. In addition, the composition may be administered via any device capable of transporting active substances to target cells.

Still another aspect of the present disclosure provides a method of screening for a therapeutic agent for autism spectrum disorder, the method including the steps of (a) treating quiescent neural stem cells with a candidate therapeutic agent for autism spectrum disorder; and (b) measuring dormancy of the neural stem cells.

The method may include the step of determining the candidate as a therapeutic agent for autism spectrum disorder when dormancy is reduced as a result of comparing dormancy of the neural stem cells of the step (a) with that of neural stem cells of a control not treated with the candidate.

The terms “dormancy of quiescent neural stem cells”, “autism spectrum disorder”, and “treatment” are as described above.

In the screening method, the dormancy of the step (b) may be to measure any one or more of (i) H3K4 trimethylation level and (ii) activity level of histone lysine methyltransferase of neural stem cells.

The (i) H3K4 trimethylation level may be measured using a method known in the art. Examples of methods of directly determining the H3K4 trimethylation level include chromatin immunoprecipitation (ChIP), Western blot, immunohistochemistry, immunocytochemistry, ELISA, etc. As another example, it may be achieved by measuring the activity level of an enzyme involved in H3K4 methylation, for example, methyltransferase or demethylase.

The (ii) activity level of histone lysine methyltransferase may be measured using a method known in the art. As a specific example, it may be achieved by measuring the expression levels of any one or more of the genes encoding Kmt2a, Kmt2b, Kmt2c, Kmt2f, Kmt2g, ASH, and Sc/Sp proteins, more specifically by measuring the expression levels of any one or more of KMT2A and KMT2C.

The expression levels of KMT2A and KMT2C may be measured by RT-PCR, competitive RT-PCR, real-time RT-PCR, RNase protection assay (RPA), Northern blotting, and mRNA expression level measurement such as DNA chips, but are not limited thereto.

The screening method may be performed in vivo or in vitro, and is not particularly limited thereto. The candidates may be known substances or novel substances, and screening may be performed on a large scale through, for example, plant extracts or chemical libraries. Through this, it is possible to find an agent capable of treating autism spectrum disorder by inhibiting dormancy of stem cells.

Still another aspect of the present disclosure provides a composition for diagnosing autism spectrum disorder, the composition including an agent capable of measuring dormancy of quiescent neural stem cells.

The agent capable of measuring dormancy of quiescent neural stem cells may be an agent capable of measuring any one or more of (i) H3K4 trimethylation level and (ii) activity level of histone lysine methyltransferase of neural stem cells. The measurement of the H3K4 trimethylation level and the activity level of histone lysine methyltransferase is as described above.

The composition for diagnosing may be provided in the form of a kit, but is not limited thereto.

Still another aspect of the present disclosure provides a method of providing information for diagnosis of autism spectrum disorder, the method including the steps of (a) measuring dormancy of quiescent neural stem cells in an individual suspected of developing autism spectrum disorder; and (b) comparing the measured level with that in a normal individual.

The measurement of the dormancy of quiescent neural stem cells of the step (a) may be measurement of any one or more of (i) H3K4 trimethylation level and (ii) activity level of histone lysine methyltransferase of neural stem cells. This is as described above.

Still another aspect of the present disclosure provides a method of preparing an autism spectrum disorder animal model, the method including the step of increasing dormancy of quiescent neural stem cells in an animal excluding humans.

Specifically, the increase of dormancy of quiescent neural stem cells may be an increase of any one or more of (i) H3K4 trimethylation level and (ii) activity level of histone lysine methyltransferase of neural stem cells.

The dormancy of quiescent neural stem cells, the increase of H3K4 trimethylation level and the increase of activity level of histone lysine methyltransferase, and autism spectrum disorder are as described above.

Still another aspect of the present disclosure provides use of the dormancy inhibitor of quiescent neural stem cells in the prevention or treatment of autism spectrum disorder.

Still another aspect of the present disclosure provides use of the agent capable of measuring dormancy of quiescent neural stem cells in the diagnosis of autism spectrum disorder.

Hereinafter, the present disclosure will be described in more detail with reference to Examples and Experimental Examples. However, these Examples and Experimental Examples are only for illustrating the present disclosure, and the scope of the present disclosure is not intended to be limited by these Examples and Experimental Examples.

### Example 1. Finding of Abnormal Activity of Super Quiescent Neural Stem Cells (qNSCs) in Autism Mouse Model

In order to identify the abnormal neurodevelopmental pattern that causes autism and to investigate the cause, a Shank3-deficient mouse model, which is a representative autism animal model, was used.

To identify abnormal neurodevelopmental patterns in the brains of Shank3-deficient autism mice, transcriptome analysis was performed in the subventricular zone (SVZ) of brains in control mice and Shank3-deficient mouse by scRNA-seq.

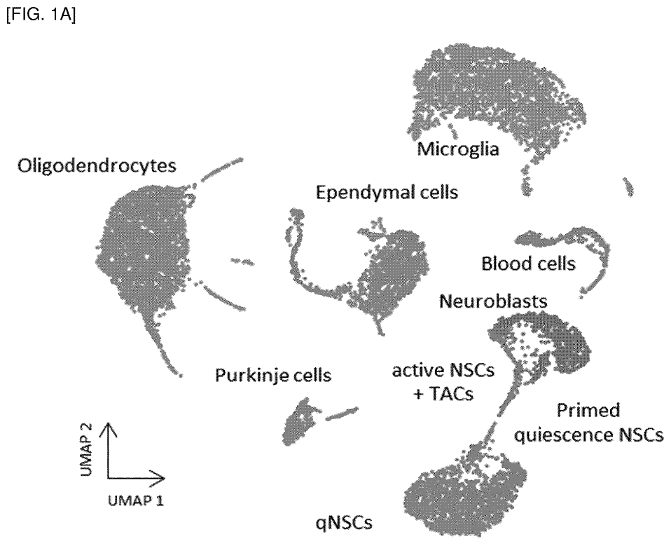

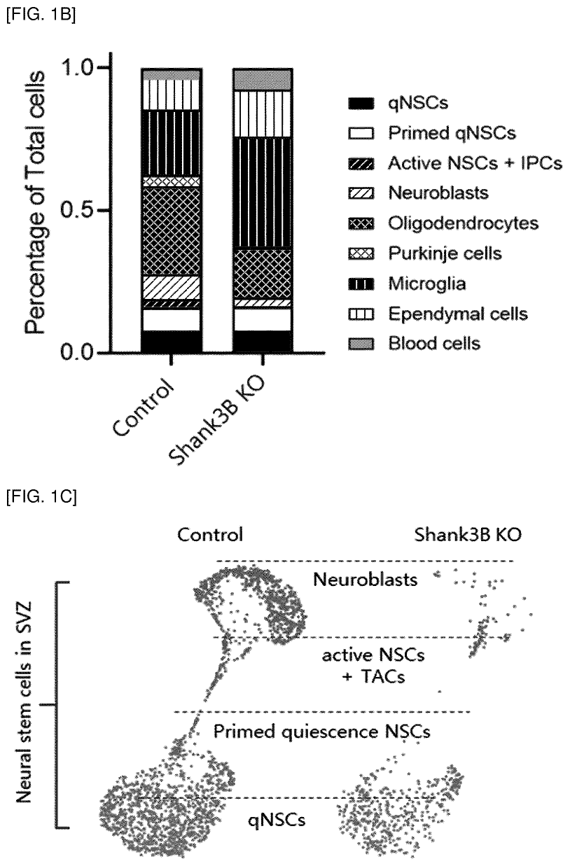

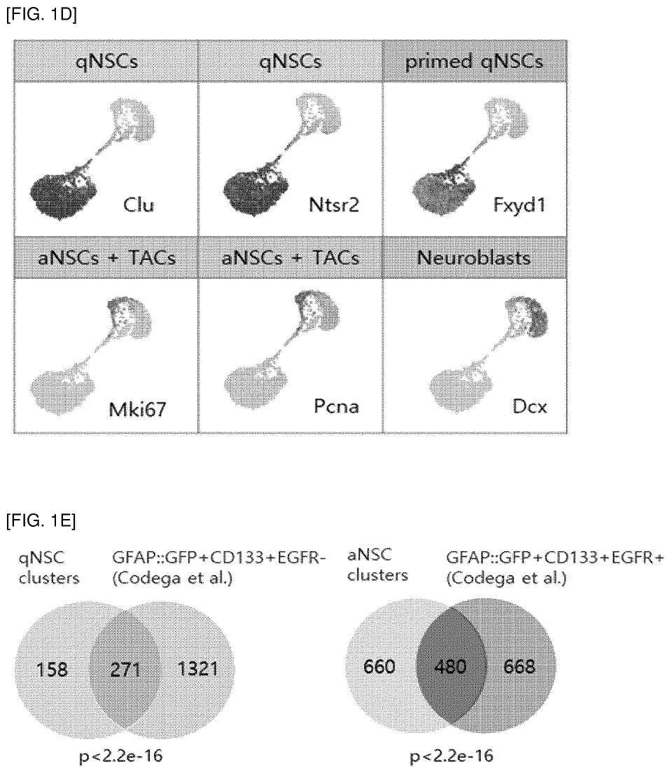

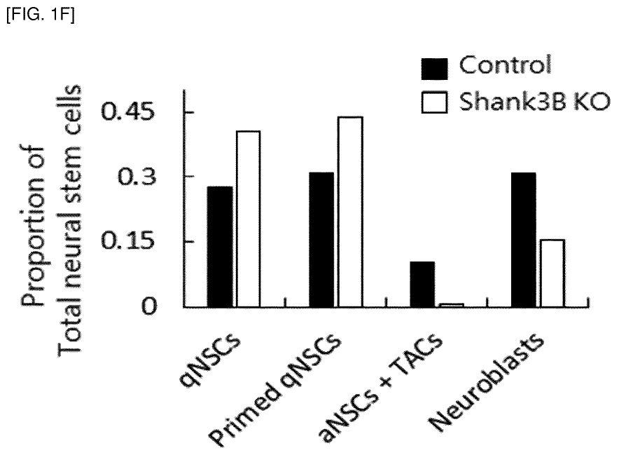

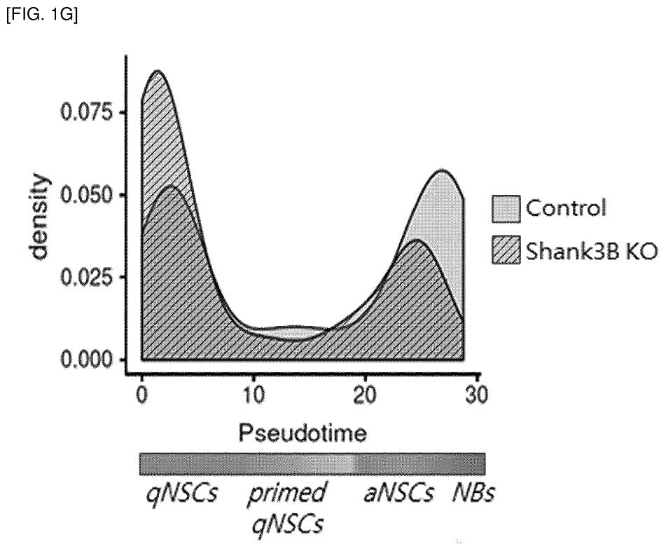

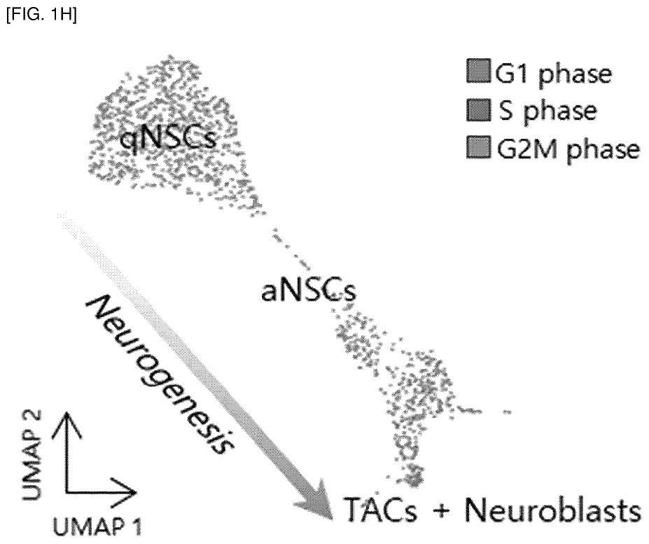

As a result, 7,368 cells and 2,708 cells were obtained from the SVZs of control and Shank3-deficient mice, respectively, and the cell distribution was shown by combining respective experimental groups on a UMAP (uniform manifold approximation and projection) plot (FIG. 1A). In addition, the identity of the cell group was revealed through the cell group-specific marker gene (FIG. 1B). Neural stem cells were grouped into four categories based on markers specific to each cell group. 1) Quiescent neural stem cells (qNSCs) expressing Ntsr2 and Clu; 2) primed quiescent neural stem cells (primed qNSC) expressing Fxyd1; 3) active neural stem cells (aNSC) expressing Mki67 and Pcna and transit-amplifying cells (TAC); and 4) neuroblast (NB) expressing Dcx (FIGS. 1C and 1D). In addition, it was confirmed that the expression profiles of qNSC and aNSC were significantly consistent with previous papers (FIG. 1E).

In particular, it was found that the total number of neural stem cells was significantly reduced in the brains of Shank3-deficient mice (FIG. 1F). As a result of pseudotemporal analysis by transcriptome analysis of clusters of neural stem cells under Shank3-deficient conditions, it was confirmed that frequency of the least differentiated NSCs, such as qNSC and primed qNSC, markedly increased in the absence of Shank3, and thus the number of differentiated NSCs actually decreased, and accordingly, overall neuronal development was delayed or inhibited (FIG. 1G). Additionally, changes in the cell cycle of NSCs during Shank3-deficient neurogenesis were further examined, and as a result, compared to the control, most of the quiescent neural stem cell groups were in the G1/G0 phase, active NSCs were in a transient state, and TACs and neuroblasts were in the S and G2M phase (FIGS. 1H and 1I).

These results show that normal neurodevelopment is not well induced due to the abnormally enhanced activity of the super quiescent neural stem cells in the Shank3-deficient representative autism mouse model, indicating that the activity of super quiescent neural stem cells is a new factor inducing autism.

### Example 2. Investigation of Abnormal Activity of Quiescent Neural Stem Cells During Neurogenesis in Autism Mice

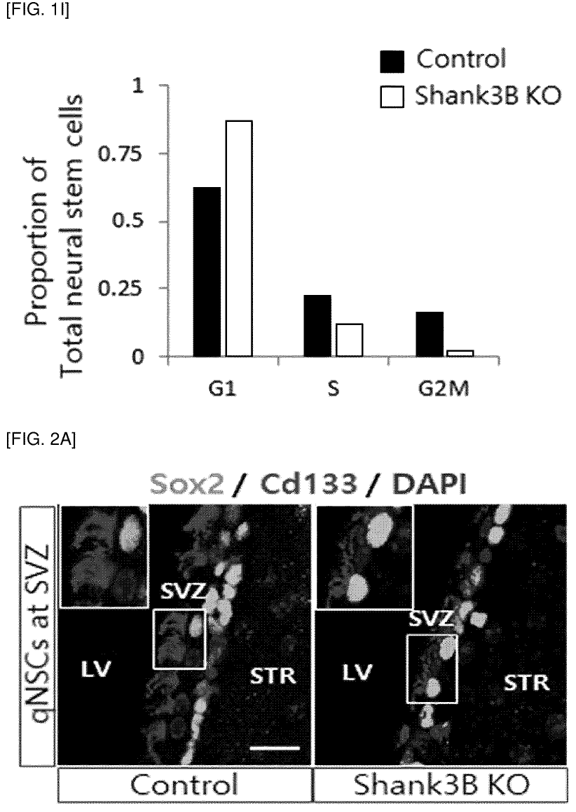

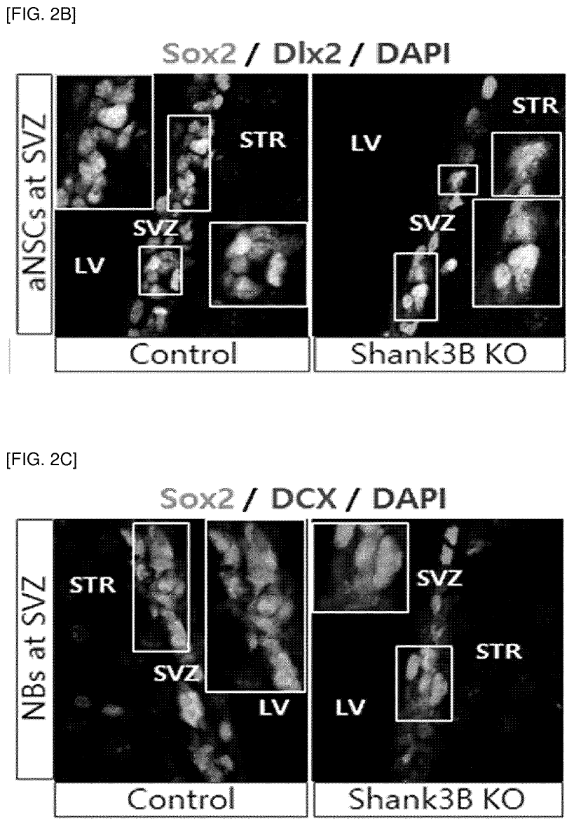

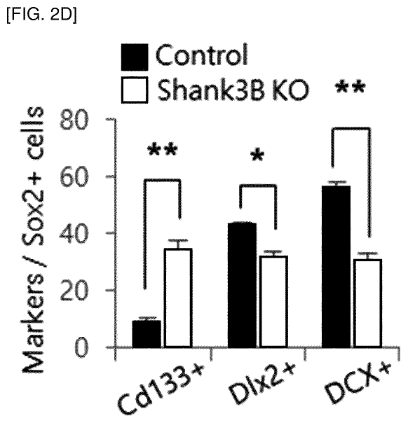

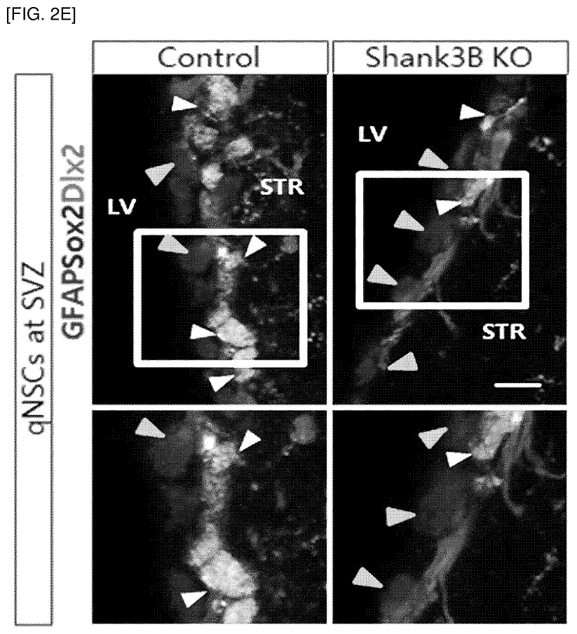

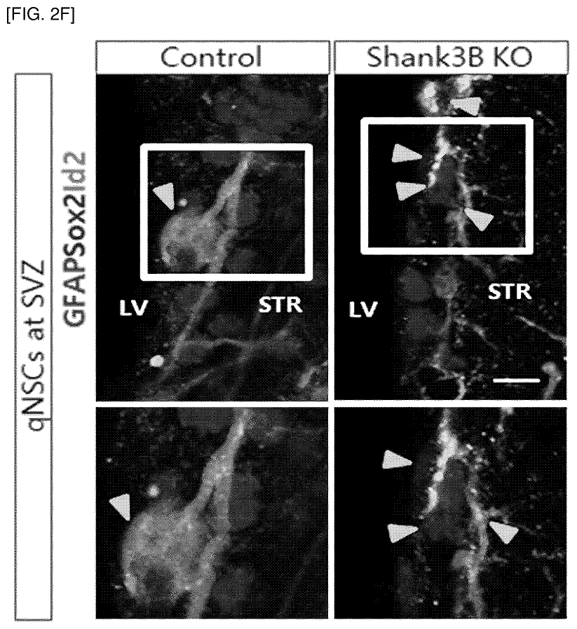

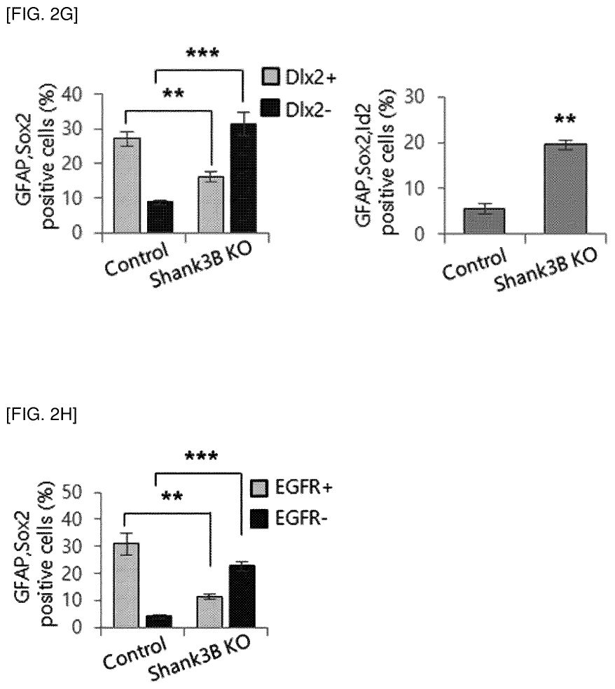

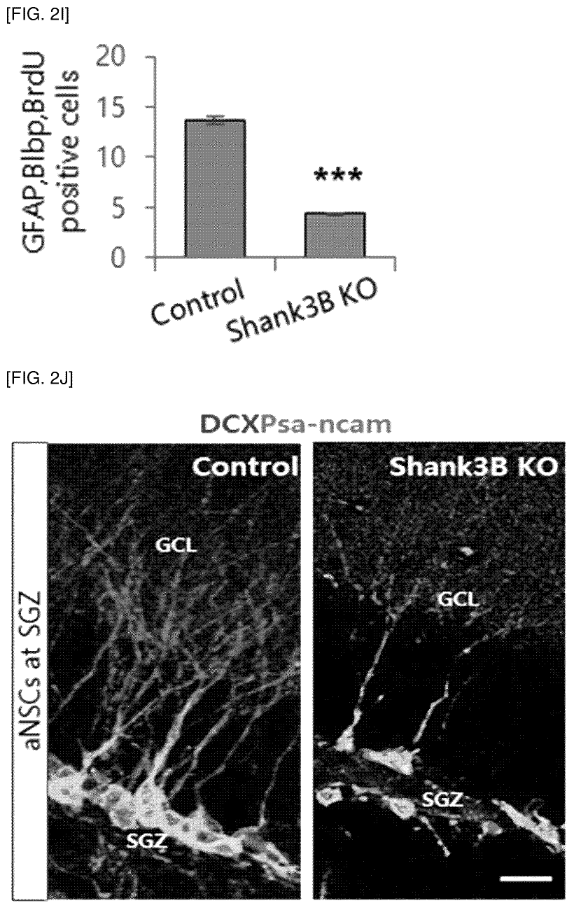

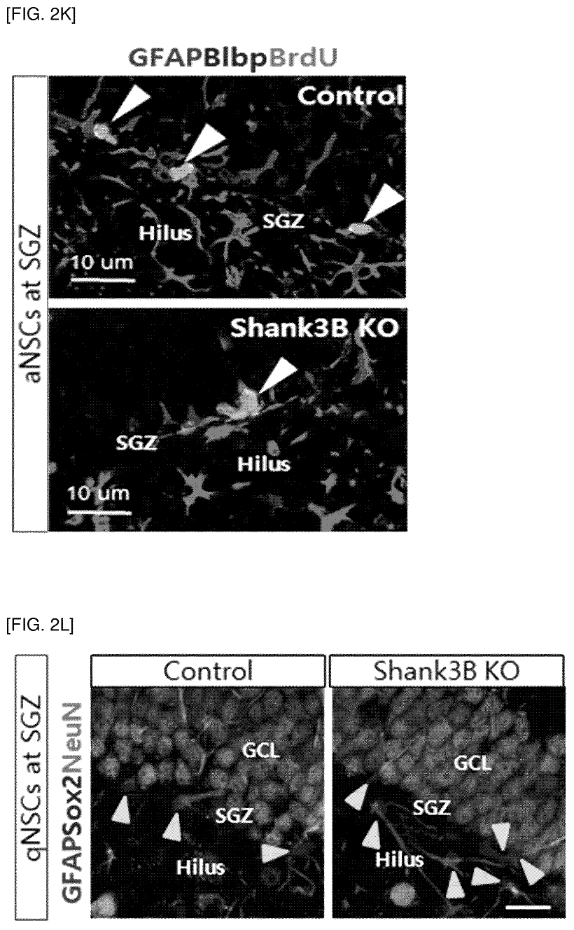

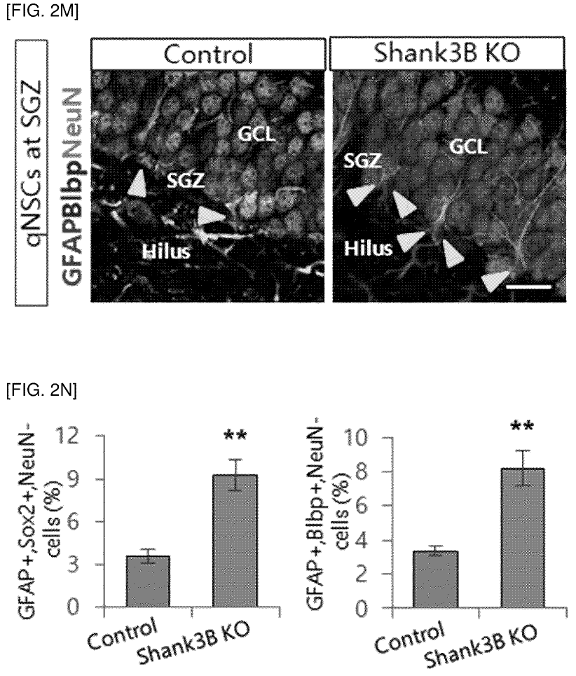

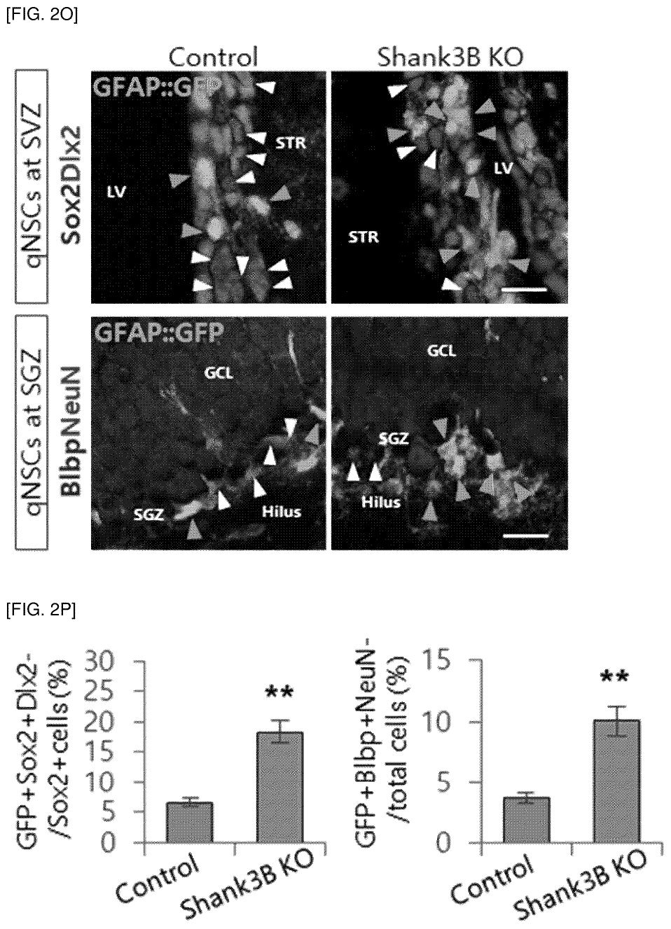

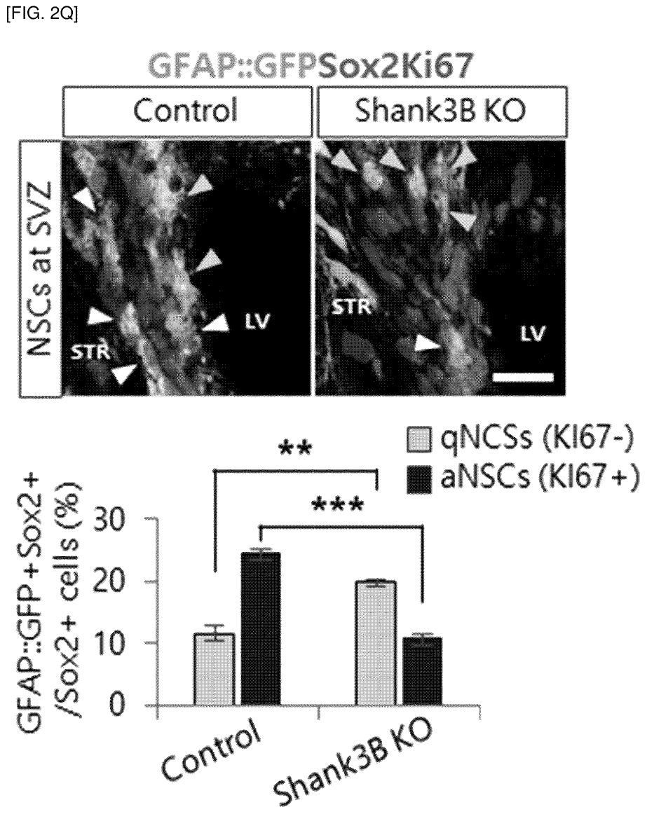

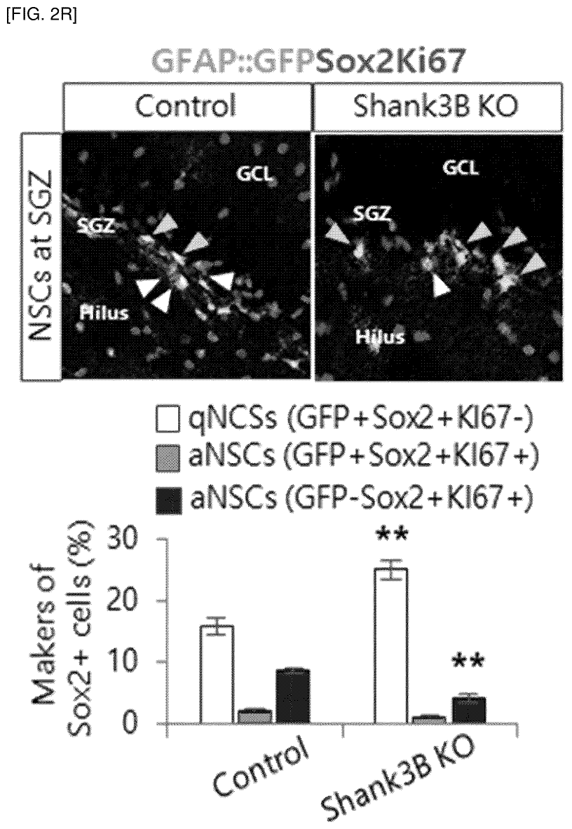

To investigate abnormal neurogenesis in the brains of Shank3-deficient mice, the neurogenesis process in the SVZ and subgranular zone (SGZ) of the brains of Shank3-deficient mice was analyzed by immunofluorescence analysis. Consistent with the results of Example 1, the number of Sox2+,CD133+ quiescent neural stem cells (qNSCs) greatly increased in the Shank3-deficient SVZ, whereas the number of Sox2+ and Dlx2+ neural stem cells (active NSCs) and the number of Sox2+ and DCX+ neuroblasts greatly decreased (FIGS. 2A to 2D).

In addition, a significant increase in GFAP+,Sox2+,Dlx2−, GFAP+,Sox2+,EGFR−, and GFAP+,Sox2+,Id2+ qNSCs was observed during neurogenesis in Shank3-deficient mice (FIGS. 2E to 2H). Consistent with these results, the number of DCX+, PSA− NCAM+, and GFAP+,Blbp+,BrdU+ active NSCs decreased in the SGZ of the brains of Shank3-deficient mice (FIGS. 21 to 2K), and a significant increase in GFAP+,Sox2+ and GFAP+,Blbp+,NeuN− qNSCS was observed (FIGS. 2L to 2N). These results demonstrate abnormal neurogenesis in the Shank3-deficient autistic brain.

To add qNSC function during neurogenesis in the Shank3-deficient mice, Shank3-deficient mice were prepared using transgenic mice in which expression of GFP was regulated by the human GFAP promoter. Consistent with the above results, GFAP::GFP+,Sox2+,Dlx2− and GFAP::GFP+,Blbp+,NeuN− qNSCs significantly increased in the brains of Shank3-deficient mice (FIGS. 2O to 2P). In addition, the number of GFAP::GFP+,Sox2+,Ki67+ active NSCs significantly decreased, whereas the number of GFAP::GFP+,Sox2+,Ki67− qNSCs significantly increased during Shank3-deficient neurogenesis (FIGS. 2Q to 2R).

These results indicate that the normal neurodevelopment is not well induced due to the abnormally enhanced activity of super quiescent neural stem cells in the autism mouse model.

### Example 3. Finding of Targets that Control Abnormal Dormant State of Quiescent Neural Stem Cells in Brain Neurogenesis Stage in Autism Mouse Model

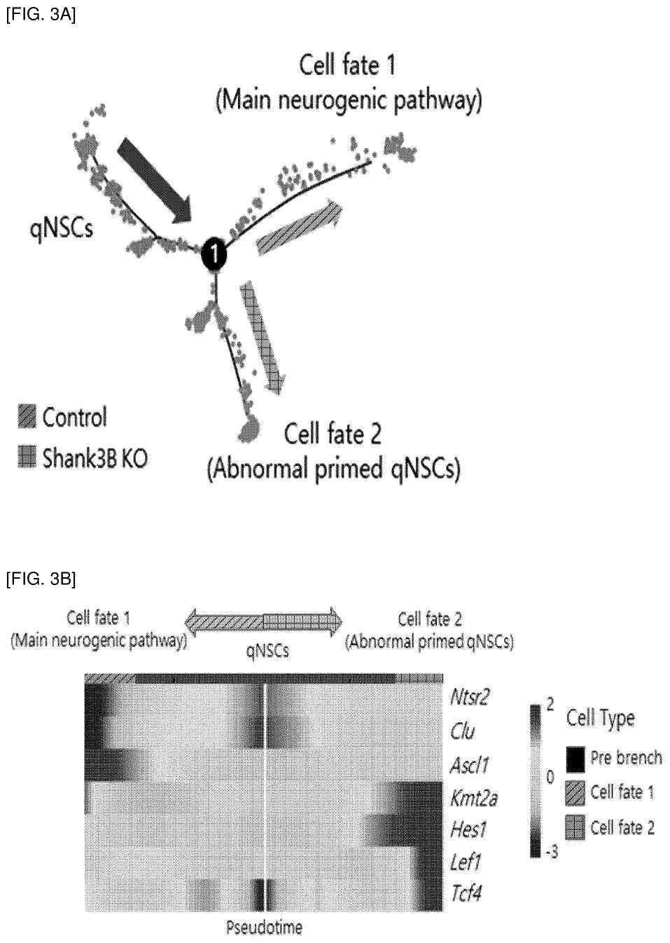

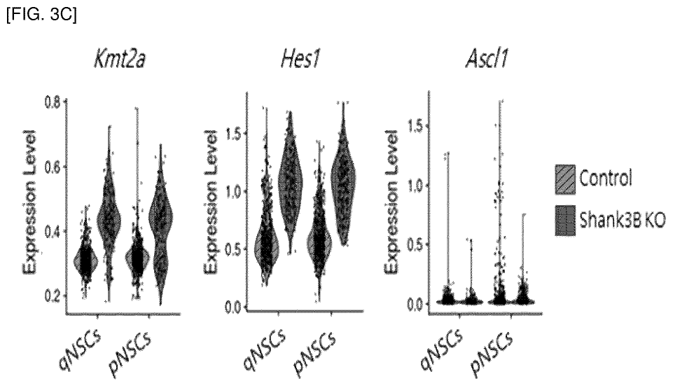

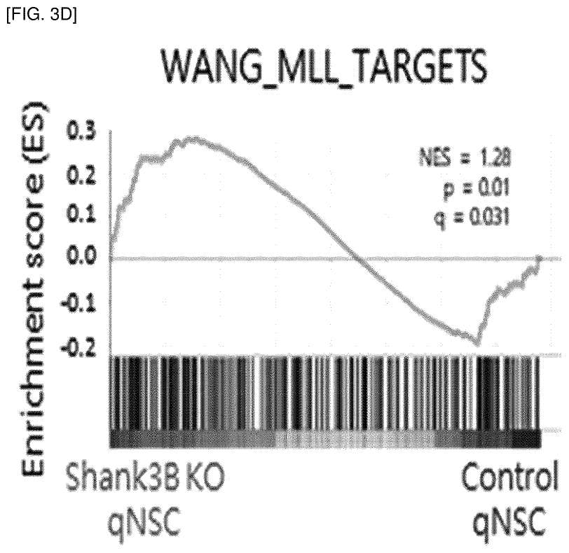

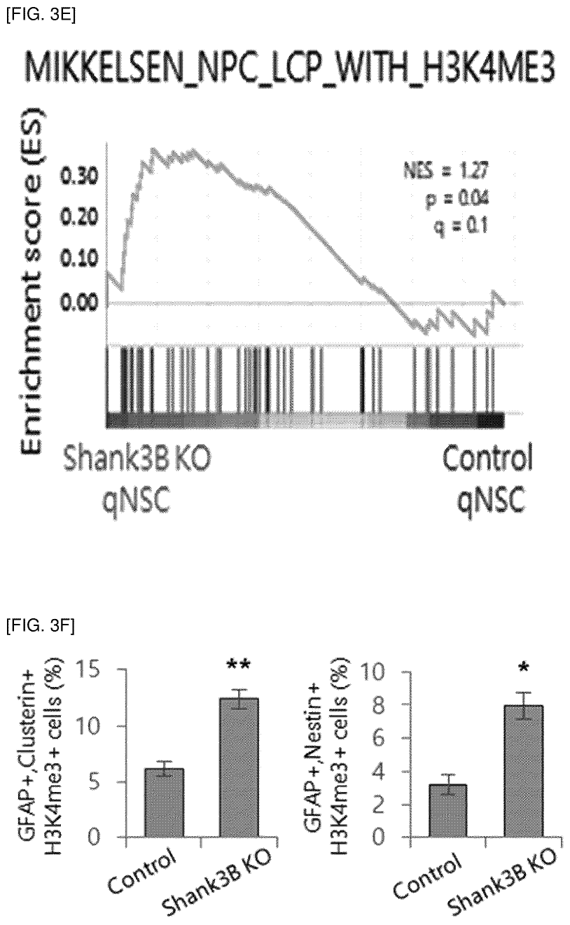

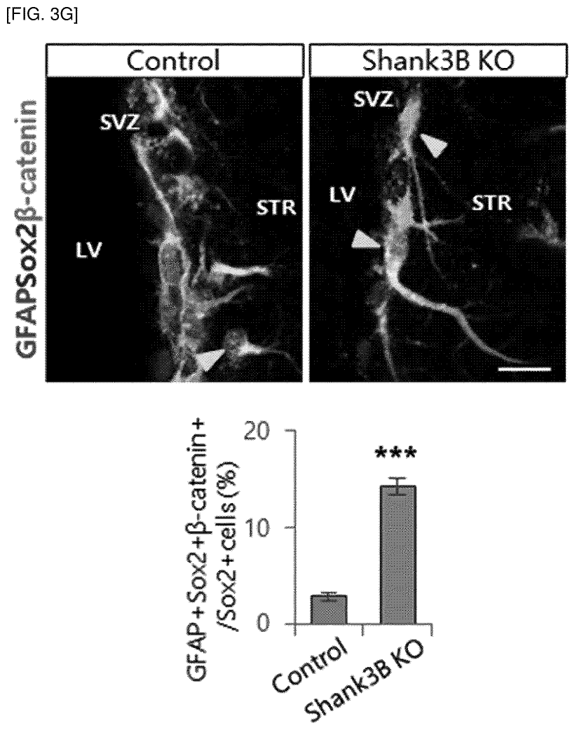

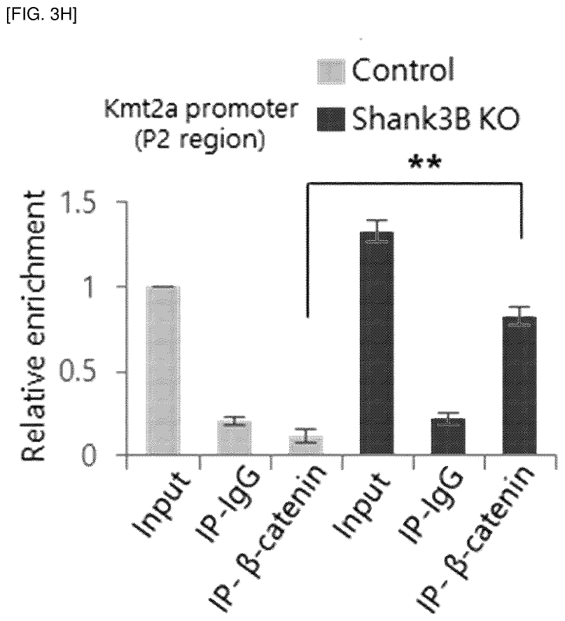

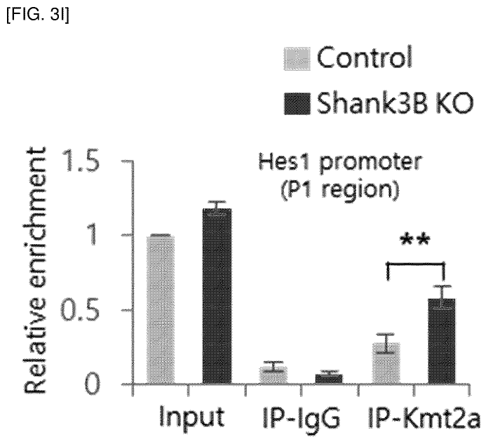

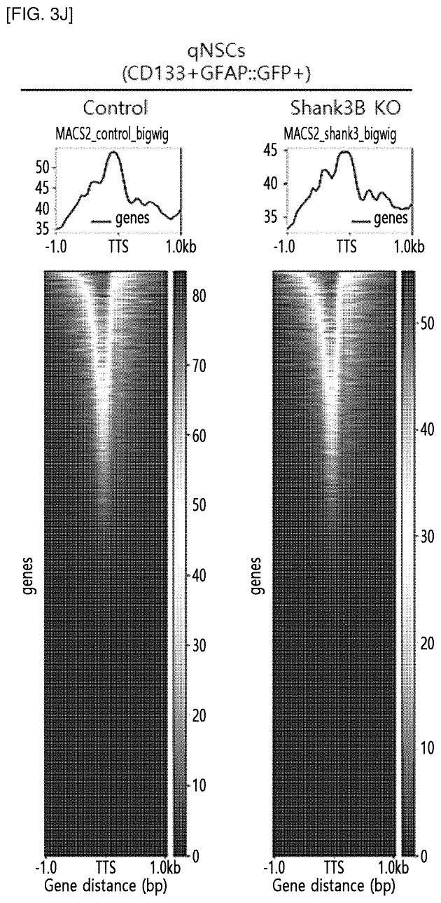

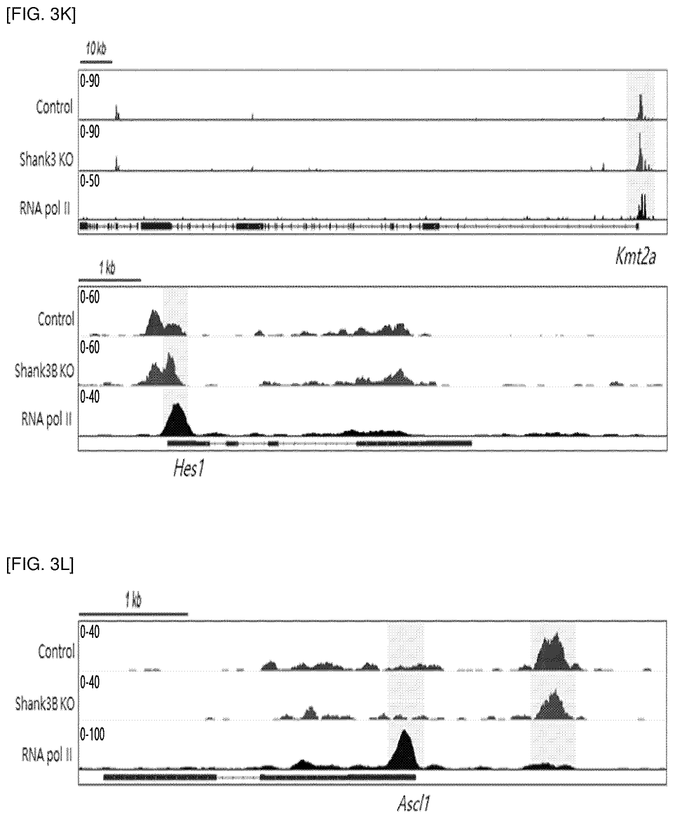

Next, pseudotemporal analysis was performed to analyze a mechanism that increases the dormant state of qNSCs in the brains of Shank3-deficient autism mice. Trajectory analysis revealed that primed qNSCs (denoted “Cell fate 1”) of qNSCs polarized at one end of the pseudo timescale, but aberrant qNSCs (denoted “Cell fate 2”) formed separate branches, suggesting that there is another neurogenic pathway of Shank3-deficient neurogenesis (FIG. 3A). Among various genes, seven genes with gene expression differences due to qNSCs, partially differentiated primed qNSCs, and abnormal qNSC branches were shown as a heat map (FIG. 3B). The primed qNSC (Cell fate 1) group that induces normal neurodevelopment showed high Ascl1 expression and low Ntsr2 and Clu expression, while the abnormal qNSC (cell fate 2) group showed a tendency to decrease Ntsr2 and Clu expression, but the expression was not completely abolished as in normal primed qNSCs (FIG. 3B). In addition, the abnormally partially differentiated primed NSCs were found to have high Hes1 gene expression (FIG. 3B). Expression of the histone lysine methyltransferase Kmt2a as well as Hes1 gene was also specifically increased in Shank3-deficient primed qNSCs (FIG. 3C). It was confirmed that the target genes to which KMT2 binds were considerably similar to the genes expressed in Shank3-deficient qNSCs. Further, GSEA analysis confirmed that genes with high binding of H3K4me3 in neural progenitor cells (NPCs) were considerably similar to Shank3-deficient qNSCs genes (FIGS. 3D and 3E). This indicates that Shank3 deficiency may increase H3K4 trimethylation that may be induced by KMT2A (FIG. 3F).

Next, to investigate a subsequent mechanism by Kmt2a due to Shank3 deficiency, the binding between Shank3 and β-catenin protein was examined in qNSCs. As a result of immunoprecipitation and Western blot analysis, the interaction between β-catenin and Shank3 was confirmed, and nuclear translocation of β-catenin was increased in Shank3-deficient qNSC (FIG. 3G). Additionally, ChIP-PCR analysis showed that the binding ratio of β-catenin in the Kmt2a promoter region (P2 region) was markedly increased in Shank3-deficient qNSCs, suggesting that β-catenin translocated into the nucleus binds to the Kmt2a promoter in Shank3-deficient conditions (FIG. 3H). Next, ChIP-PCR analysis was performed to examine whether or not Kmt2a protein binds to the Hes1 promoter in the absence of Shank3, and it was found that kmt2a binds to the Hes1 promoter region to regulate gene expression (FIG. 3I).

To further understand the transcriptional dynamics of Shank3-deficient qNSCs, transposase-accessible chromatin (ATAC-seq) analysis was performed to characterize open chromatin regions in Shank3-deficient qNSCs. Chromatin accessibility around the transcription start site (TSS) region was very similar regardless of Shank3 status (FIG. 3J), but that in the promoters of Kmt2a and Hes1 was increased in Shank3-deficient qNSCs (FIG. 3K). In contrast, the chromatin accessibility of Ascl1 was reduced (FIG. 3L), demonstrating that high Hes1 expression suppresses Ascl1 expression in neurogenesis, thereby maintaining the dormant state of qNSCs.

Accordingly, these results imply that H3K4me3 deposition by KMT2A is associated with increased activity of the Hes1 promoter, resulting in autism-related abnormal neurogenesis through the induction of pathological qNSC dormancy in the brains of Shank3-deficient mice.

### Example 4. Therapeutic Technique for Deficits in Social Behaviors of Autism Mice Through Regulation of Kmt2a Activity in Quiescent Neural Stem Cells

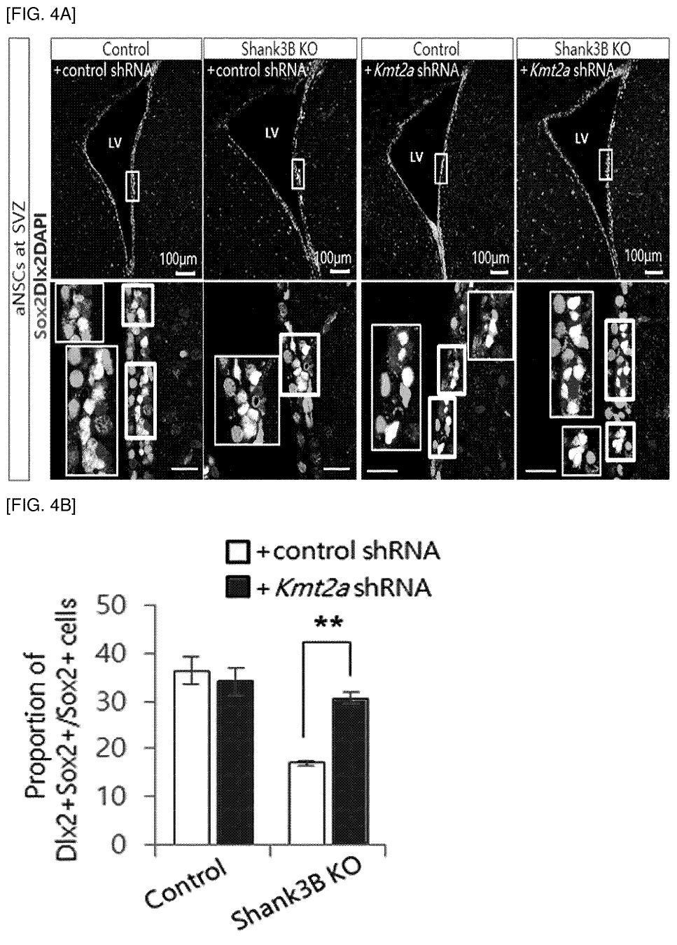

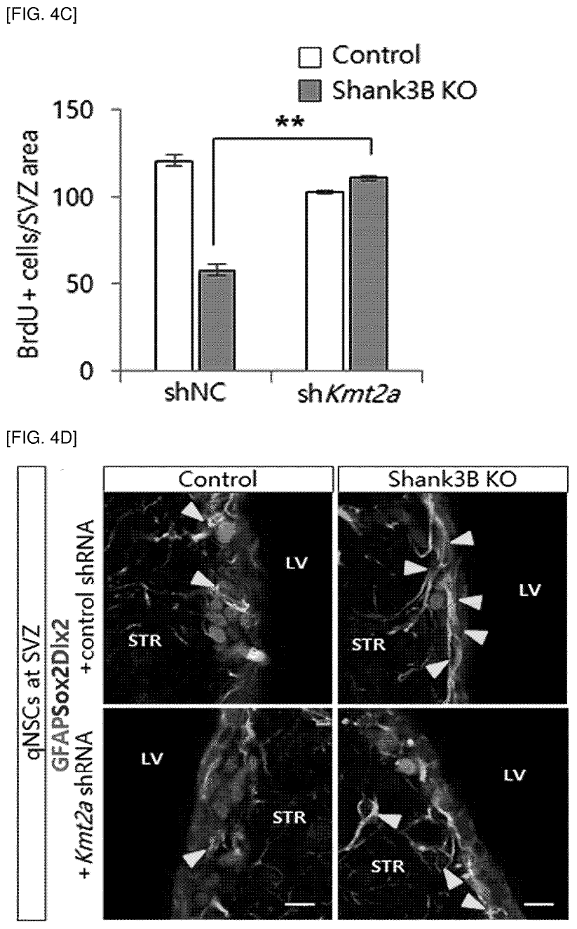

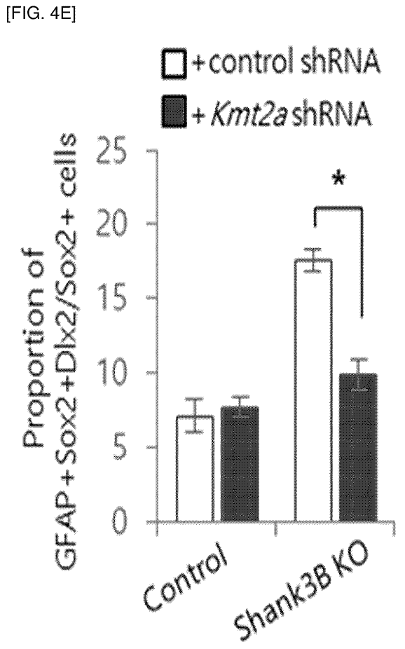

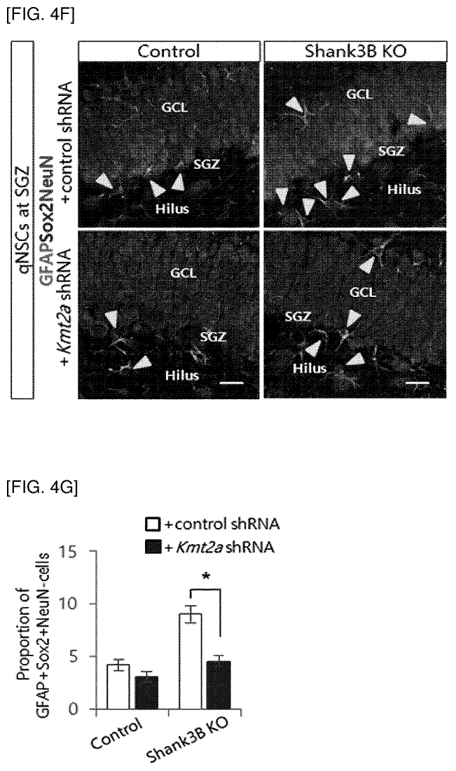

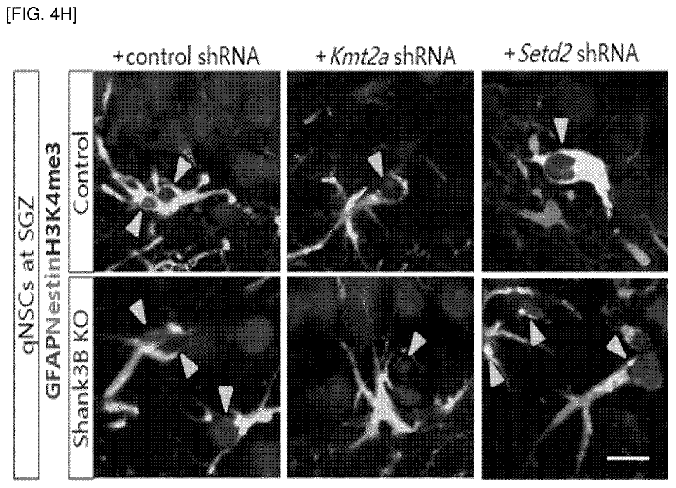

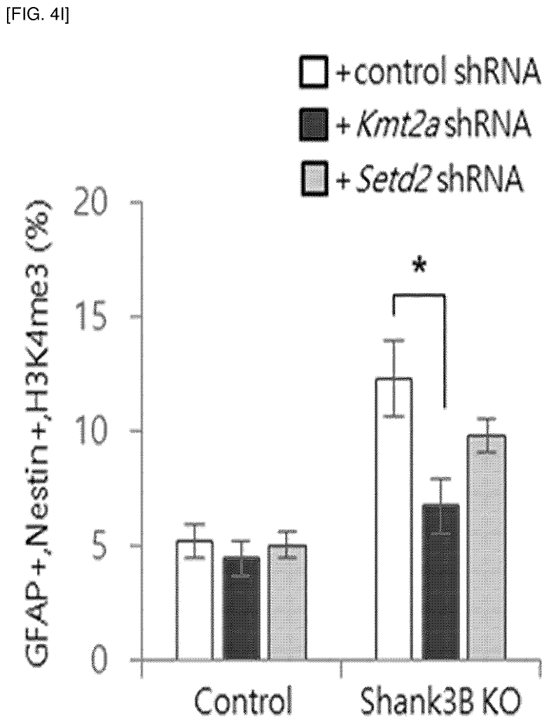

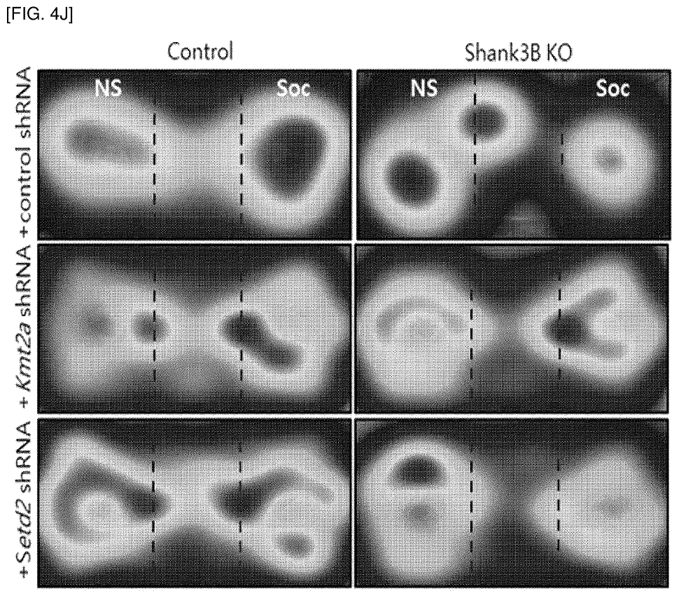

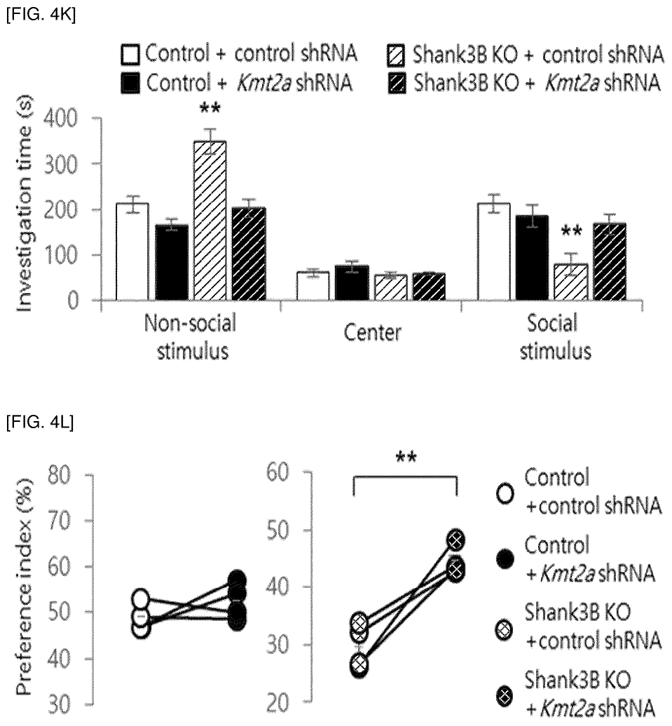

To determine whether or not Kmt2a functionally contributes to the Shank3-related autism phenotype and adult neurogenesis, shRNA against Kmt2a mRNA was designed (Target sequence: CTGATTCGCAAACCAATATTT) and injected into the SVZ and SGZ of the brains of 8-week-old Shank3-deficient mice. Seven days after injection of Kmt2a shRNA into the SVZ, a significant increase in the number of Sox2+,Dlx2+ active NSCs was observed in the neural stem cells of the brains of Shank3-deficient mice (FIGS. 4A and 4B). In addition, upon Kmt2a knockdown, BrdU-positive cells significantly increased in the neural stem cells of Shank3-deficient mice (FIG. 4C). Consistent with this, a proportion of GFAP+,Sox2+,Dlx2− qNSCs among the total Sox2+ cells was decreased upon Kmt2a shRNA treatment (FIGS. 4D and 4E), and the reduced GFAP+, Sox2+, and NeuN− qNSCs were also observed during neurogenesis in the neural stem cell region (SGZ) of the hippocampus of Shank3-deficient mice (FIGS. 4F and 4G). Collectively, these data demonstrated that Kmt2a knockdown may effectively restore Shank3-associated abnormal dormancy in quiescent neural stem cells and neurogenesis. Additionally, it was confirmed that Kmt2a knockdown resulted in a significant decrease in H3K4me3 cells, which were increased in GFAP+,Nestin+ qNSCs of Shank3-deficient mice (FIGS. 4H and 41).

Next, it was analyzed whether or not Shank3-associated autistic social/behavioral abnormalities during neurogenesis in the adult brain may be alleviated by Kmt2a knockdown. The results of a three-chamber social interaction behavior test showed that Kmt2a shRNA treatment dramatically increased social interaction time and social preference in Shank3-deficient mice, as compared to control, whereas both control shRNA and Setd2−shRNA-treated mice maintained asocial behaviors (FIGS. 4J to 4L). Overall, these findings demonstrate that regulation of dormancy through Kmt2a shRNA in quiescent neural stem cells may control aberrant neurogenesis to ameliorate social behavioral deficits in autism.

### Example 5. Analysis of Restoration of Dormancy and Therapeutic Effect of Social Behavior Deficit Through Regulation of Abnormal Activity of Quiescent Neural Stem Cells by OICR-9429 Drug

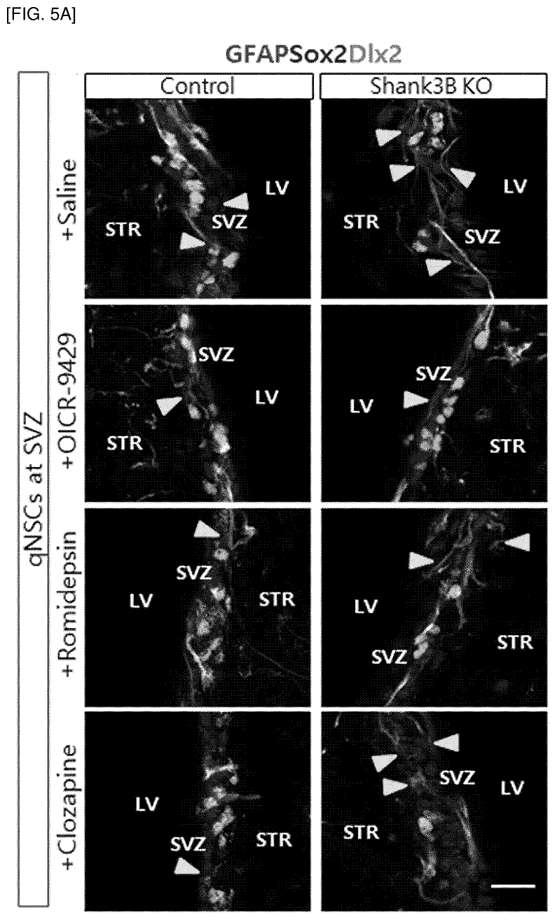

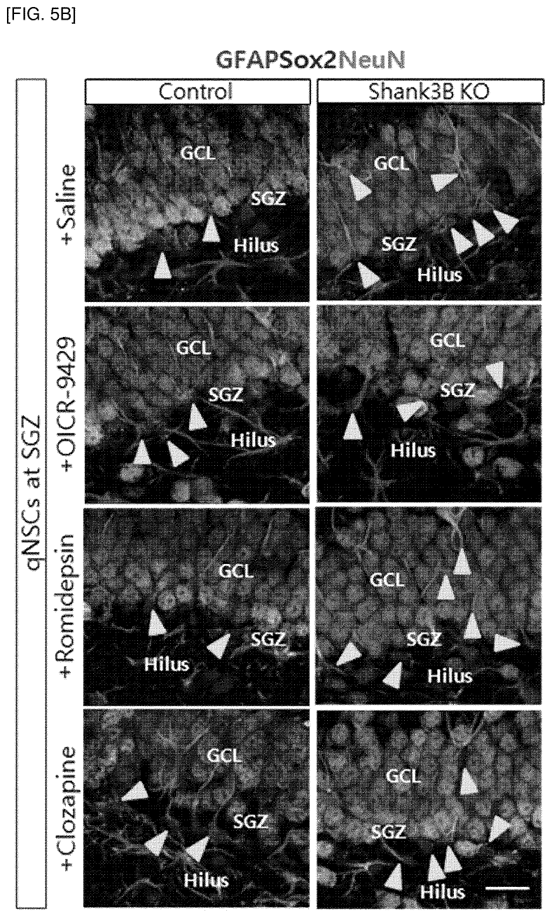

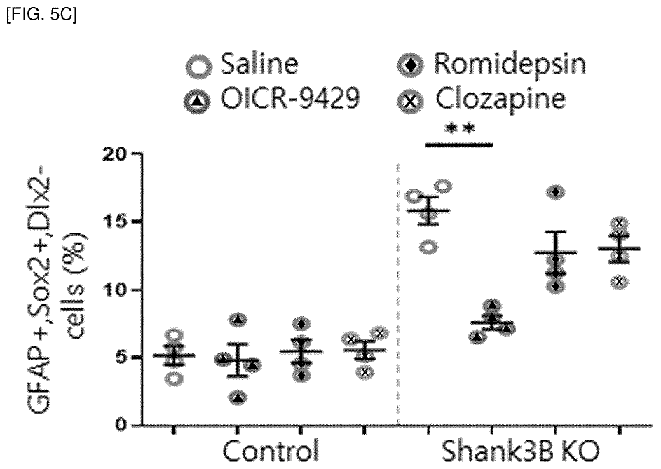

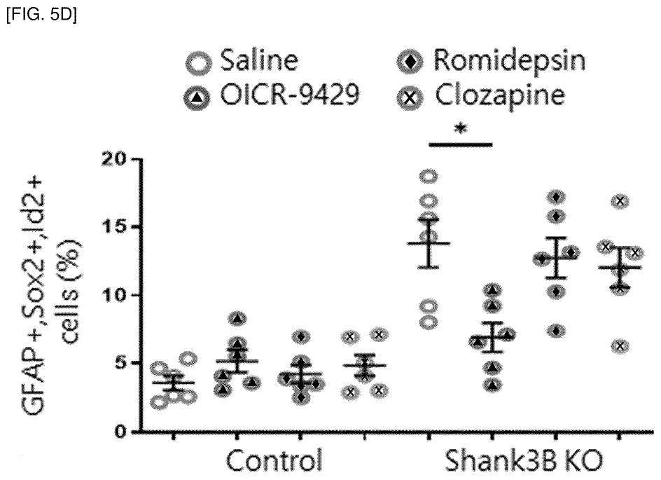

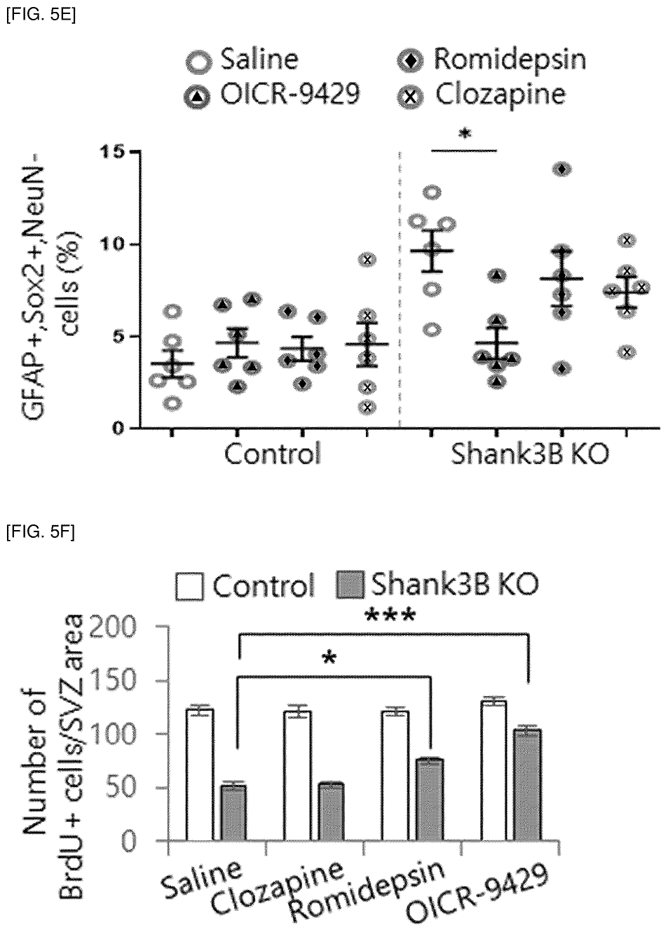

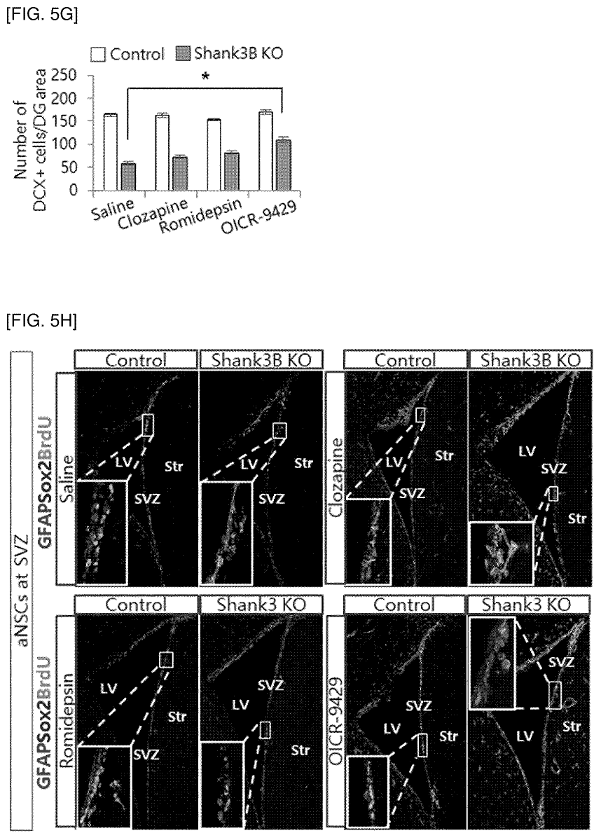

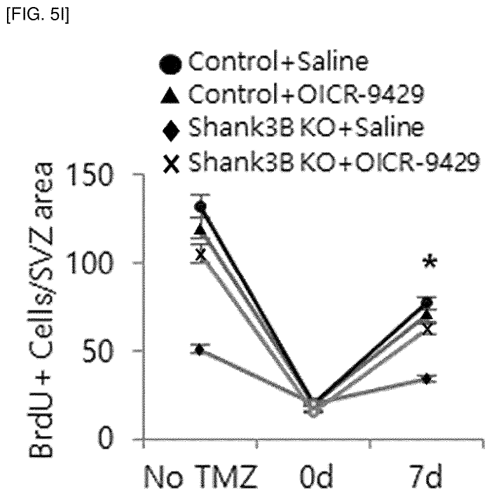

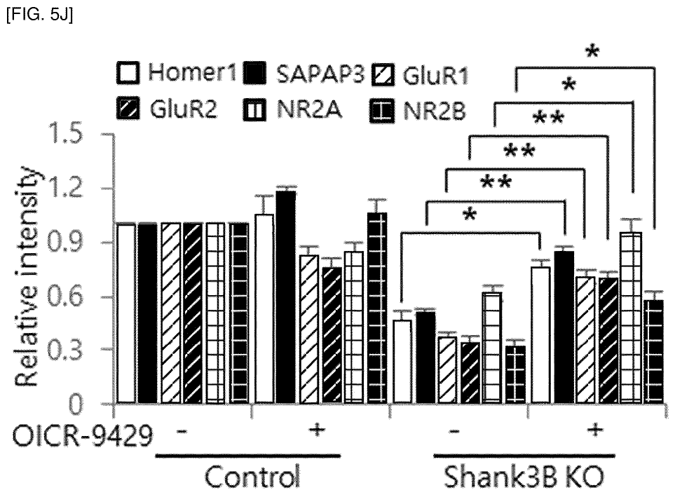

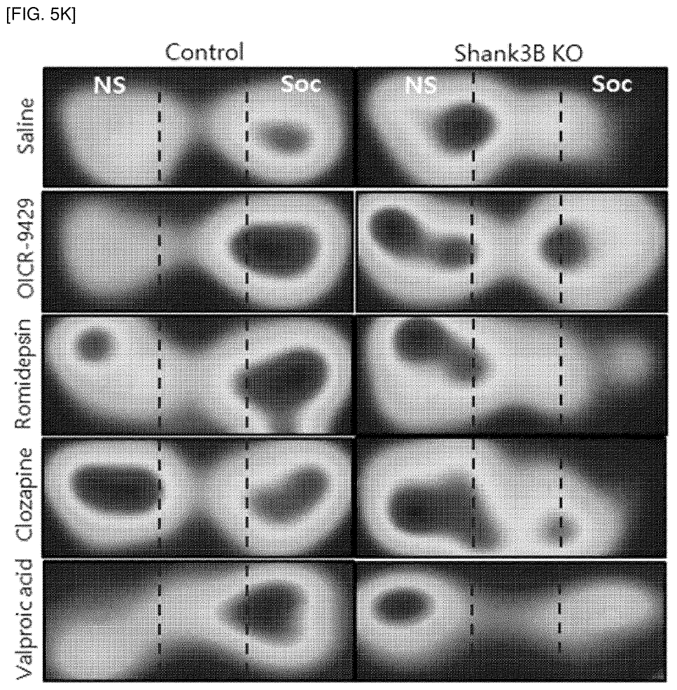

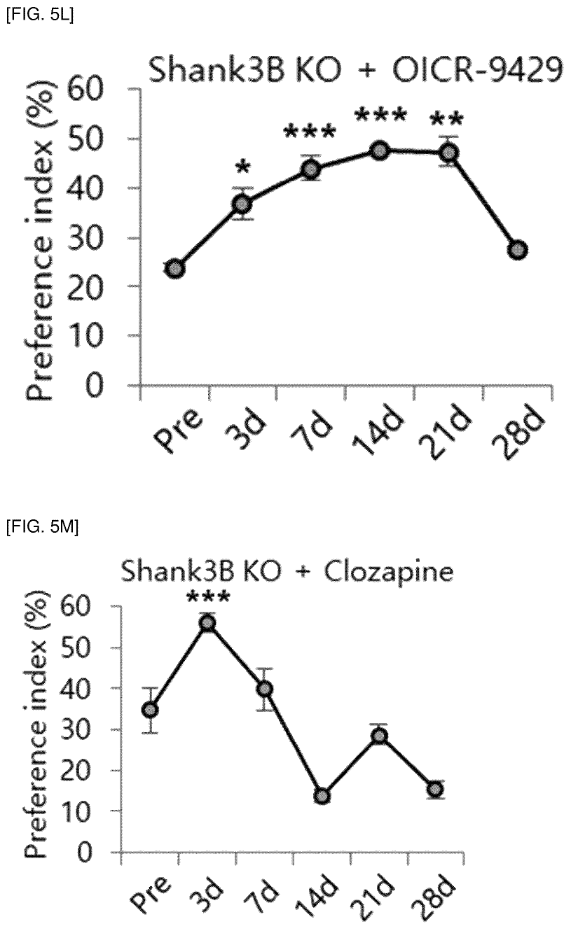

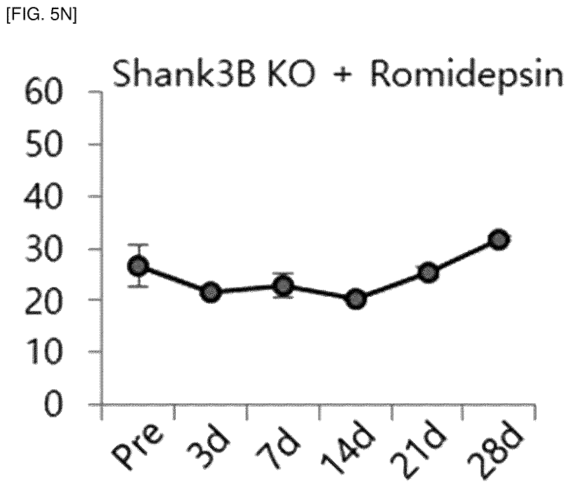

To provide a drug-based therapeutic strategy for autism, Shank3-deficient mice were treated with OICR-9429, which is an antagonist inhibiting H3K4 trimethylation by KMT2A, and the effect was investigated. 2 weeks after OICR-9429 administration, it was found that the number of GFAP+,Sox2+,Id2+ and GFAP+,Sox2+,Dlx2− qNSCs significantly decreased, and HDAC inhibitor romidepsin or serotonin antagonist clozapine, known as a mechanism drug, had no effect (FIGS. 5A, 5C, and 5D). Similarly, OICR-9429 injection reduced the number of GFAP+,Sox2+,NeuN− qNSCs during hippocampal neurogenesis in Shank3-deficient mice (FIGS. 5B and 5E). Moreover, OICR-9429 greatly increased the number of rapidly dividing cells labeled with BrdU, whereas other antipsychotic drugs showed no effect (FIGS. 5F and 5H). Finally, as a result of treatment with OICR-9429, the number of DCX-positive neuroblasts increased in the brains of Shank3-deficient mice (FIG. 5G). Taken together, these results show that OICR-9429 restored normal adult neurogenesis in autism mice by blocking the Kmt2a activity and inhibiting H3K4me3 to suppress abnormal qNSC dormancy.

Next, to investigate the functional role of OICR-9429 on qNSC activity, analysis was performed by treatment with temozolomide (TMZ). After 7 days of TMZ injection, BrdU-positive cells were significantly recovered by OICR-9429 treatment to a level close to that of control mice (FIG. 51), indicating that new proliferating cells are effectively generated by suppressing dormancy of qNSCs. In addition, expression of synaptic proteins Homer1 and SAPAP3 and glutamate receptor subunits GluR1, GluR2, NR2A, and NR2B was significantly restored in Shank3-deficient mice treated with OICR-9429, indicating that OICR-9429 may restore synaptic function and development, which are autism phenotypes.

In addition, to investigate the therapeutic effect of OICR-9429, a social interaction behavioral test was performed on Shank3-deficient mice injected with various neuropsychiatric drugs. It was confirmed that Shank3-deficient mice treated with OICR-9429 spent significantly more time participating in social stimuli (i.e., similar to saline-treated control mice) (FIG. 5K); however, other antipsychotics such as romidepsin, clozapine, and VPA had no significant effect (FIG. 5K). In addition, as a result of examining the persistence of the OICR-9429 effect in the behaviors of Shank3-deficient mice, it was confirmed that the social preference index was gradually improved up to 3 weeks after injection (FIG. 5L). In contrast, the social preference index of Shank3-deficient mice administered with clozapine significantly increased during 72 hours after injection, but then rapidly decreased (FIG. 5M). Romidepsin and VPA did not improve social deficits in Shank3-deficient mice (FIGS. 5N and 5O).

Based on this, it can be seen that OICR-9429 is an effective therapeutic strategy for treating autism-related social behavioral deficits by targeting the qNSC activity.

### Example 6. Investigation of Abnormal qNSC and Neurogenesis in Human Neurons and Patient Brains with Autism Phenotypes

### Example 6-1-1. Preparation of Human iPSC-Derived Neural Stem Cells with Autism Phenotype

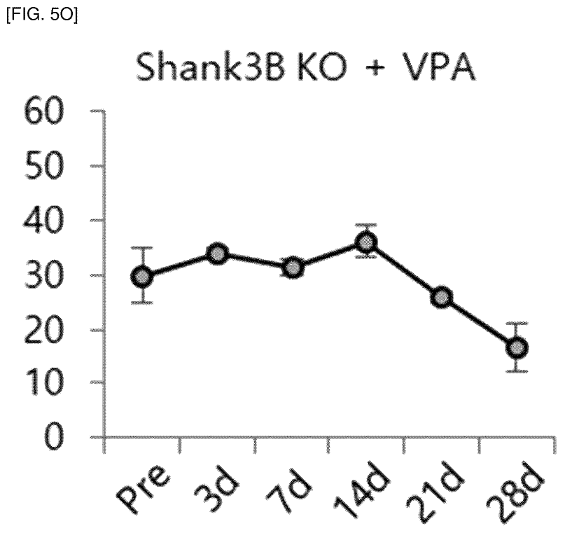

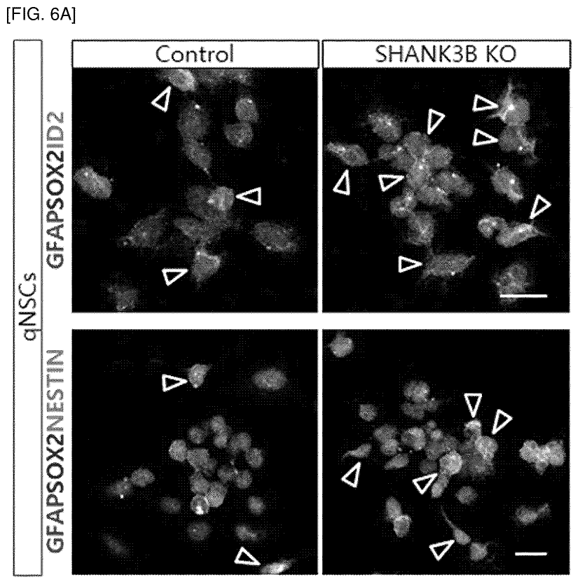

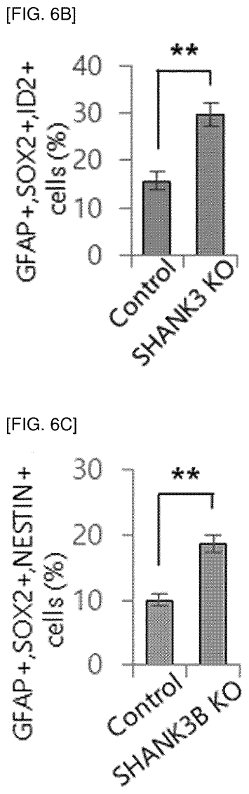

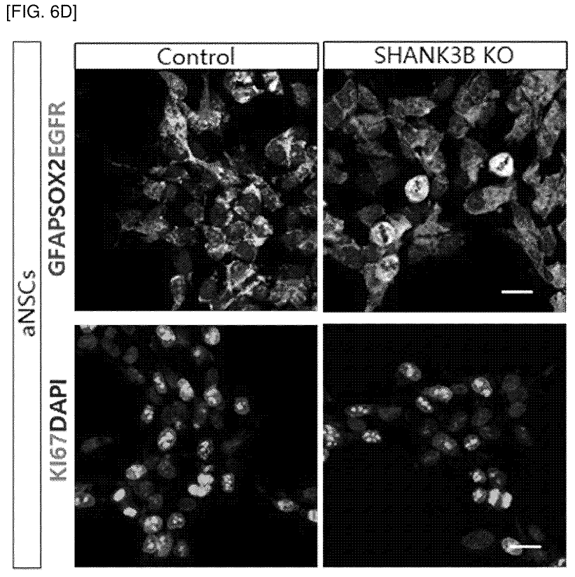

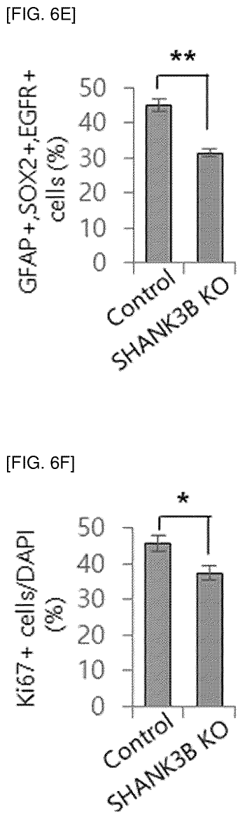

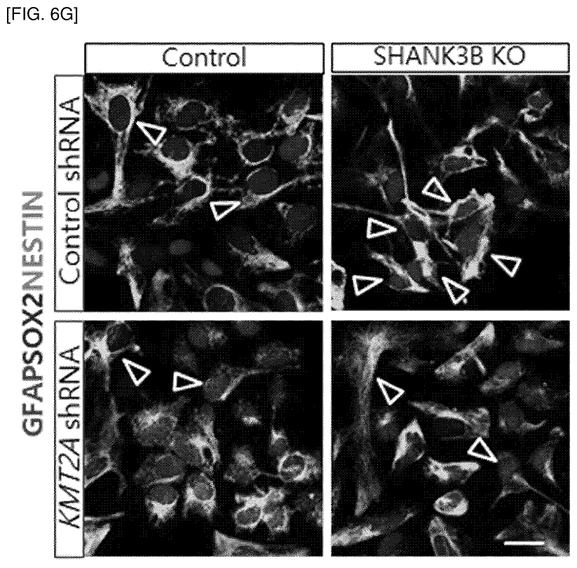

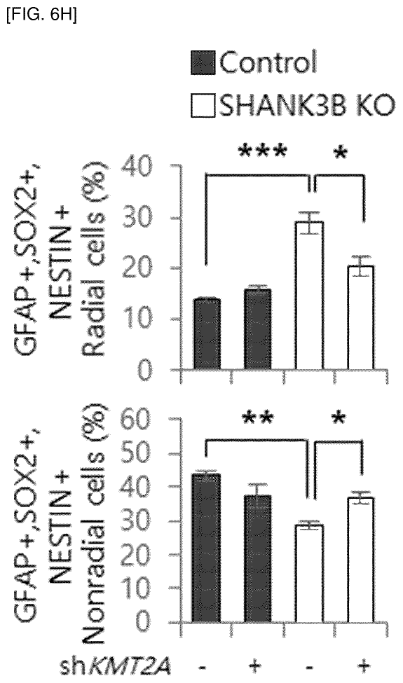

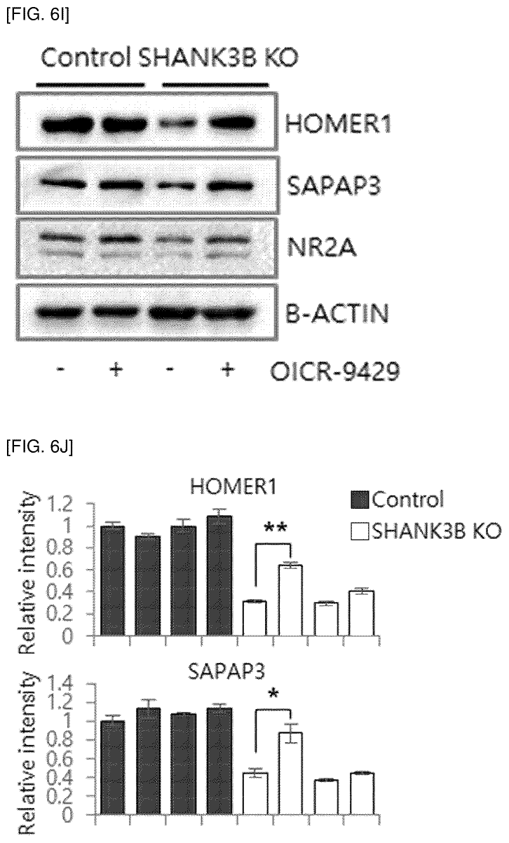

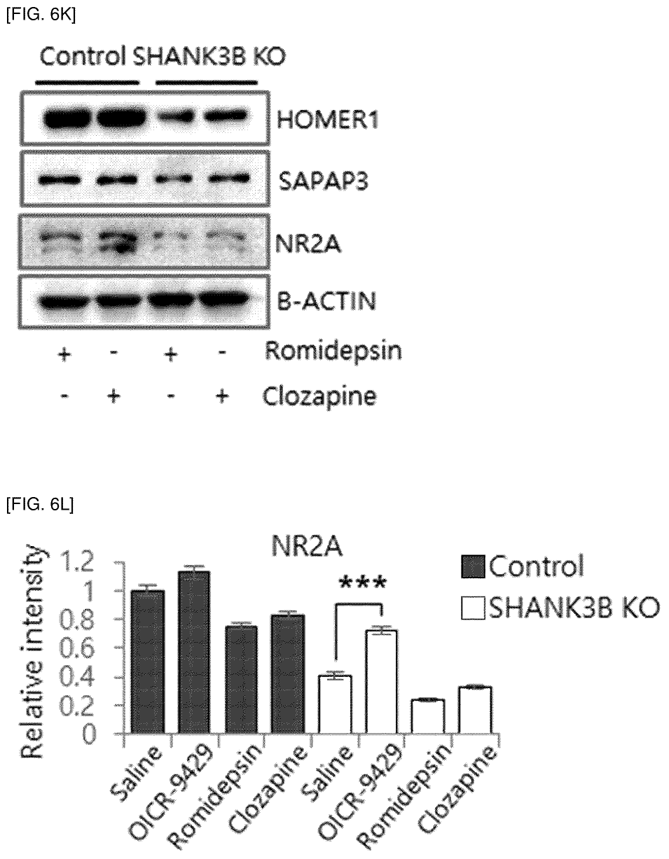

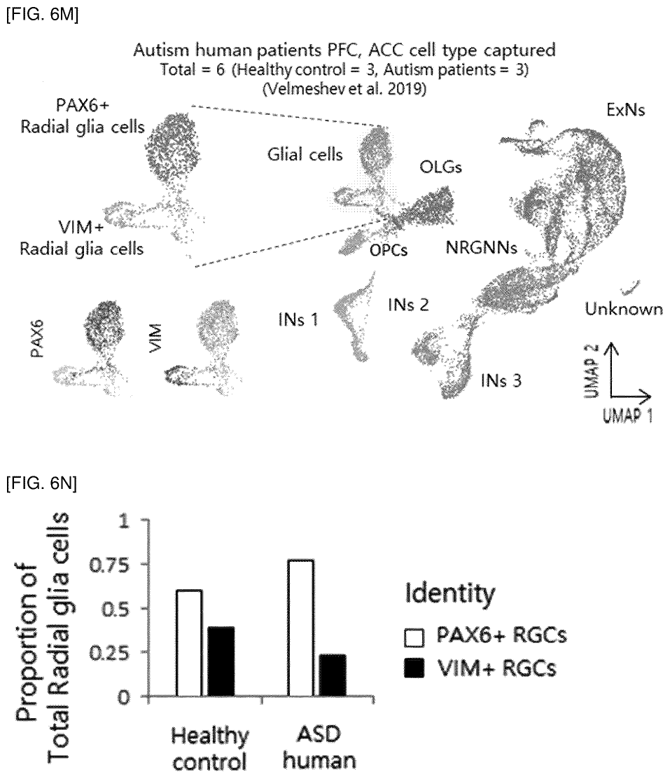

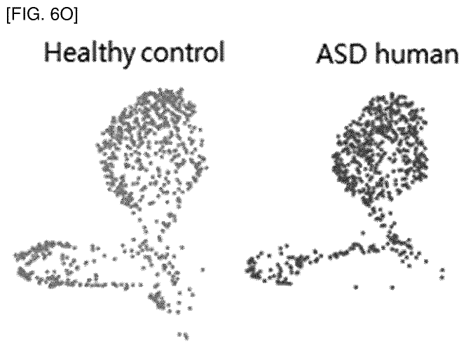

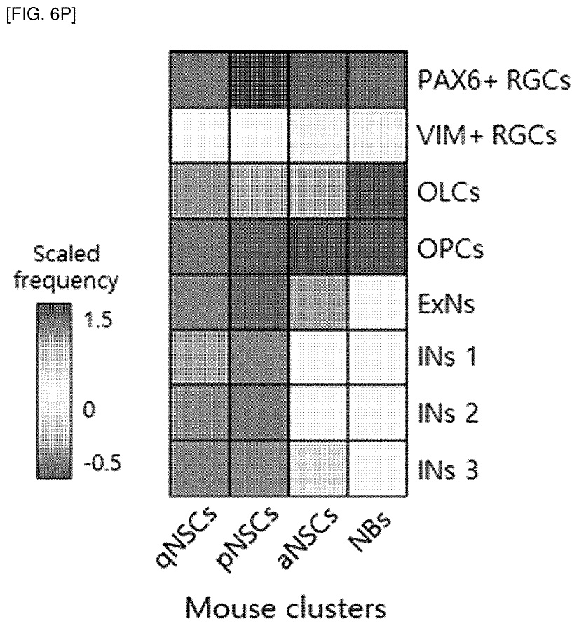

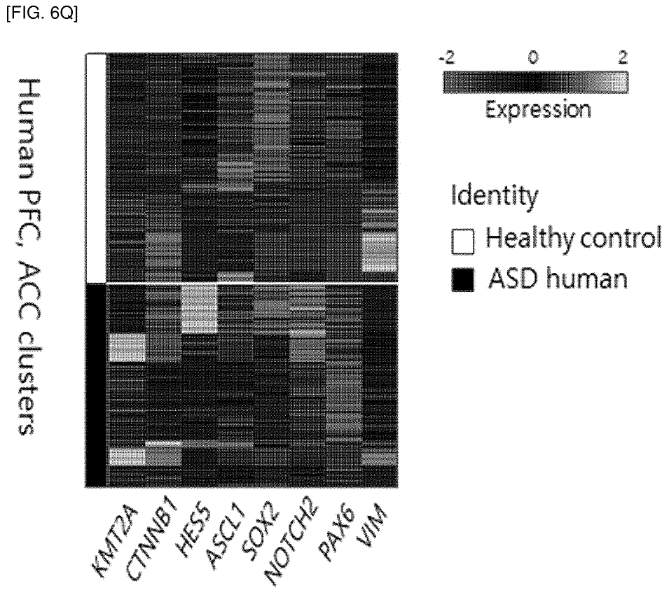

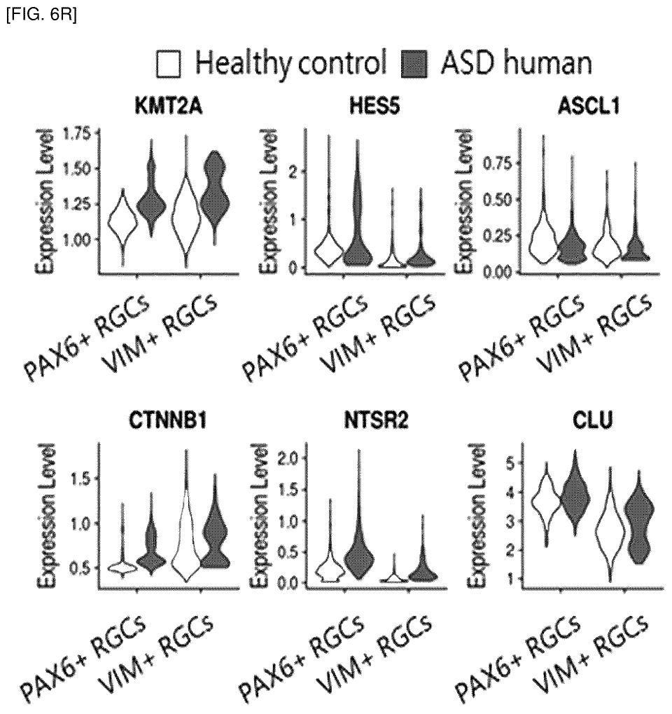

Next, neural stem cells were generated from SHANK3-deficient human iPSCs. Consistent with previous mouse data, it was confirmed that the number of GFAP+,SOX2+,NESTIN+ and GFAP+,SOX2+,I D2+ human quiescent neural stem (radial glia) cells significantly increased in the consistent SHANK3-deficient NSCs (FIGS. 6A to 6C). At the same time point, a proportion of GFAP+SOX2+EGFR+ active NSCs was significantly reduced in the absence of SHANK3 (FIGS. 6D and 6E). In addition, a proportion of Ki67+ proliferating cells was significantly reduced in SHANK3-deficient neural stem cells, as compared to the control (FIGS. 6D and 6F). These results demonstrate autism phenotypes in SHANK3-deficient human neural stem cells.

### Example 6-1-2. Investigation of Autism Treatment Effect Using Human iPSC-Derived Neural Stem Cells with Autism Phenotype

The molecular mechanisms underlying the autism phenotypes associated with SHANK3 deficiency in human iPSC-derived neural differentiation were investigated. To investigate whether or not KMT2A functionally contributes to the Shank3-deficient autism phenotype in human neural stem cells, consistent with the mouse in vivo findings mentioned above, KMT2A shRNA was transduced into SHANK3-deficient human quiescent neural stem (human radial glia) cells. As a result, upon KMT2A knockdown, a significant increase in DCX+ and Ki67+ neuroblasts was observed in SHANK3-deficient NSCs, and a proportion of GFAP+SOX2+NESTIN+ radial glia cells among the total SOX2+ cells was significantly reduced in SHANK3-deficient neural stem cells when treated with KMT2A shRNA (FIGS. 6G and 6H). This indicates that knockdown of KMT2A effectively restores neurogenesis in SHANK3-deficient cells. Additionally, it was analyzed whether or not SHANK3-deficient human radial glia cells are pharmacologically treated by OICR-9429. Five days after OICR-9429 treatment of SHANK3-deficient radial glia cells, a proportion of GFAP+,SOX2+,NESTIN+ radial glia cells with radial morphology decreased, whereas a significant decrease in a proportion of GFAP+,SOX2+,NESTIN+ radial glia cells with non-radial morphology was observed in SHANK3-deficient neural stem cells. These results indicate that inhibition of KMT2A activity inhibited the dormant state of human radial glia cells during neurogenesis. In addition, treatment with OICR-9429 greatly increased expression levels of synaptic proteins such as HOMER1 and SAPAP3 and glutamate receptor subunits GluR1 and NR2A, whereas treatment with romidepsin and clozapine did not affect the expression of these proteins in SHANK3-deficient cultures (FIGS. 61 to 6L). Taken together, these results suggest that inhibition of KMT2A activity by OICR-9429 may be a promising treatment for SHANK3-related autism.

### Example 6-2. qNSC Analysis of Brains of Autism Patients and Comparison with Mouse Models

Finally, it was confirmed whether abnormal qNSC activity and neurogenesis could be demonstrated in autism patients. First, previously published scRNA-seq data sets were reanalyzed in the brains of autism patients (n=6) (Velmeshev et al., 2019). Focusing on the PFC and ACC regions, the entire cluster containing PAX6+ and Vimentin+ radial glia cells was compared with the previously defined cluster (FIG. 6M). It was found that a ratio of PAX6+ radial glia cells to the total radial glia cells increased in the brains of these autism patients (FIGS. 6N and 6O). When mouse NSCs were compared with human radial glia cells, oligodendrocytes, oligodendrocyte precursor cells, and mature neurons, it was found that the population of human PAX6+ radial glia cells was very similar to that of mouse qNSCs and primed qNSCs, and it was demonstrated that their expression pattern was very similar to that of mouse qNSCs (FIG. 6P). In addition, significant expression of qNSC-related genes such as KMT2A, HES5, ASCL1, etc. was found in PAX6+ radial glia cells of autism patients (FIGS. 6Q and 6R).

In other words, major similarities between molecular indicators of autism found in human qNSC and Shank3-deficient autism mouse qNSC were confirmed, and the results obtained using the Shank3-deficient autism mouse model may also be applied to human autism patients. In addition, as demonstrated in the previous Examples, it can be seen that the regulation of the dormancy of qNSC of the present disclosure may be usefully applied to autism treatment not only for animals but also for humans.

### Example 7. Treatment of Social Behavioral Deficits in Autism Mice by Regulating Kmt2c Activity in Quiescent Neural Stem Cells

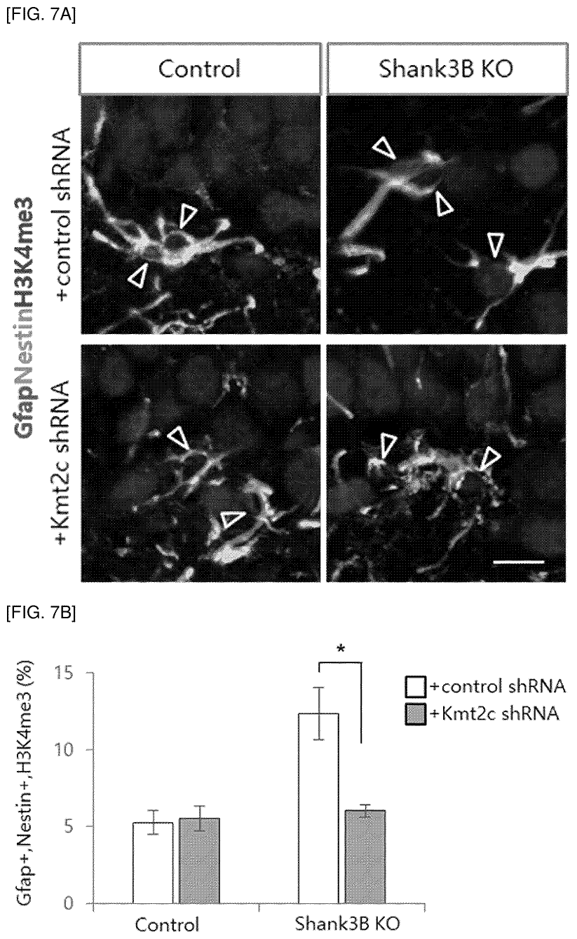

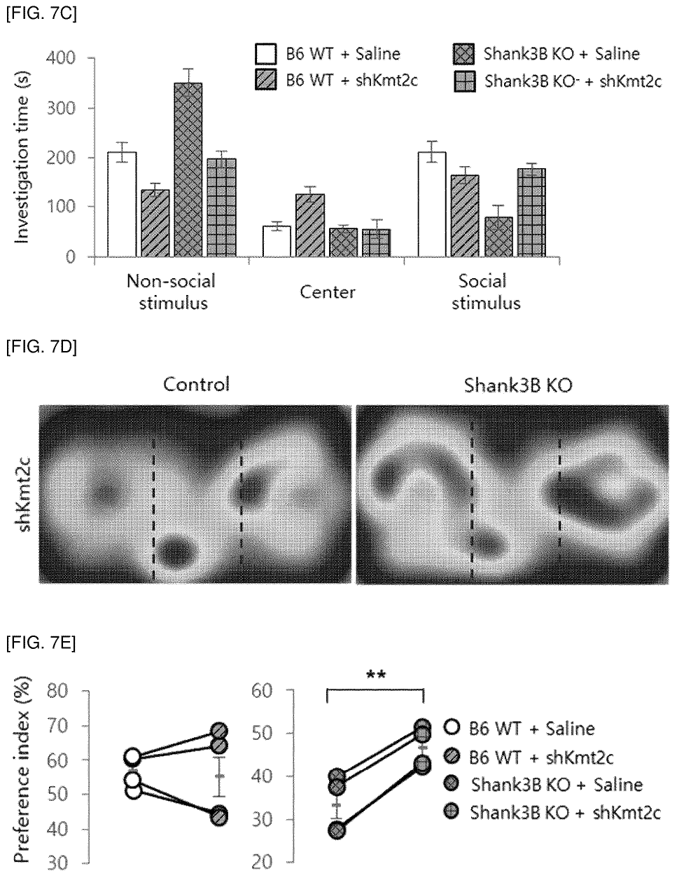

To examine whether or not Kmt2c, which is involved in H3K4 methylation, functionally contributes to the autism phenotypes, shRNA against Kmt2c mRNA was injected into the SVZ and SGZ regions of the brains of 8-week-old autism mice. As a result of immunofluorescence analysis, it was confirmed that the H3K4me3 level was decreased in GFAP+,Nestin+ qNSCs of the Kmt2c shRNA-treated group, as compared to those of control (FIGS. 7A and 7B). In addition, as a result of a three-chamber social interaction behavior analysis, it was confirmed that Shank3 KO mice administered with the control shRNA maintained asocial behavior, whereas the Kmt2c shRNA-treated group recovered social behavior (FIGS. 7C and 7D). It was confirmed that the social preference (a ratio of social stimuli/non-social stimuli) of autism mice administered with Kmt2c shRNA recovered to a level similar to that of normal mice (FIG. 7E).

Based on this, it was confirmed that the Kmt2c inhibitor may be usefully applied to treatment of autism-related social behavioral deficits.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims or equivalents of such metes and bounds are therefore intended to be embraced by the claims.

### Effect of the Invention

The present disclosure provides methods of effectively diagnosing and effectively treating autism spectrum disorder, which may also be usefully applied to screening for therapeutic agents for autism spectrum disorder.

1. A method of preventing or treating autism spectrum disorder, the method comprising inhibiting dormancy of quiescent neural stem cells (qNSCs).

2. The method of claim 1, comprising inhibition of H3K4 trimethylation in neural stem cells.

3. The method of claim 1, comprising administering an activity inhibitor of histone lysine methyltransferase.

4. The method of claim 3, wherein the histone lysine methyltransferase is one or more selected from Kmt2a and Kmt2c.

5. The method of claim 1, comprising the step of administering one or more of OICR-9429, Kmt2a inhibitor, and Kmt2c inhibitor.

6-8. (canceled)

9. A method of screening for a therapeutic agent for autism spectrum disorder, the method comprising the steps of:
(a) treating quiescent neural stem cells with a candidate prophylactic or therapeutic agent for autism spectrum disorder; and
(b) determining the candidate as a prophylactic or therapeutic agent for autism spectrum disorder, when dormancy is reduced as a result of measuring dormancy of the neural stem cells.

10. The method of claim 9, wherein the dormancy of the step (b) is measuring any one or more of an H3K4 trimethylation level and a histone lysine methyltransferase activity level in neural stem cells.

11. A method for diagnosis of autism spectrum disorder, the method comprising the steps of:
(a) measuring dormancy of quiescent neural stem cells in an individual suspected of developing autism spectrum disorder; and
(b) comparing the measured level with that in a normal individual.

12. The method of claim 11, wherein the measuring of dormancy of quiescent neural stem cells of the step (a) is measuring any one or more of (i) an H3K4 trimethylation level and (ii) a histone lysine methyltransferase activity level in neural stem cells.

13-14. (canceled)

15. A method of preparing an autism spectrum disorder animal model, the method comprising the step of increasing dormancy of quiescent neural stem cells in an animal excluding humans.

16. The method of claim 15, wherein the increasing of dormancy of quiescent neural stem cells is increasing any one or more of (i) an H3K4 trimethylation level and (ii) a histone lysine methyltransferase activity level in neural stem cells.
